(19) United States
(12) Patent Application Publication
(10) Pub. No.: US 2005/0208058 A1
(43) Pub. Date: Sep. 22, 2005

(54) Compositions and methods for modulating cell division

(76) Inventors: Stephen J. Doxsey, Princeton, MA (US); Keith Mikule, Oxford, MA (US)

Correspondence Address: FISH & RICHARDSON PC, P.O. BOX 1022, MINNEAPOLIS, MN, 55440-1022, US

(21) Appl. No.: 10/995,795

(22) Filed: Nov. 22, 2004

Related U.S. Application Data
(60) Provisional application No. 60/523,949, filed on Nov. 21, 2003.

(51) Int. Cl. C12Q 1/68; G01N 33/53; G01N 33/567; G01N 33/574; A61K 39/395

(52) U.S. Cl. 424/155.1; 435/6; 435/7.23; 514/44

(57) ABSTRACT

New methods are provided to treat cell proliferative, or other, disorders, or symptoms thereof, by modulating (e.g., inhibiting) centrosomal genes or polypeptides. These methods can include gene silencing (e.g., siRNA), antibodies, aptamers, ribozymes, antisense nucleic acids, and small molecules.

### CROSS-REFERENCE TO RELATED APPLICATIONS

This application claims priority to U.S. Provisional Application No. 60/523,949, filed on Nov. 21, 2003, the contents of which are hereby incorporated by reference in their entirety.

### TECHNICAL FIELD OF THE INVENTION

This invention relates to centrosome function, and more particularly to a centrosome damage checkpoint that can trigger a cell's withdrawal from the cell cycle.

### BACKGROUND OF THE INVENTION

Centrosomes are the major microtubule nucleating organelles in most animal cells. They nucleate and organize microtubules for spindle assembly during mitosis; and they establish microtubule arrays in interphase cells for numerous cellular functions. Centrosomes are comprised of two major structural elements, centrioles and the pericentriolar material/centrosome matrix. Each centrosome has a pair of centrioles, or microtubule barrels, that appear to organize the pericentriolar material (Bobinnec et al., J. Cell Biol., 143:1575-1589, 1998) and anchor microtubules (Chretien et al., J. Cell Biol., 120:117-133, 1997; Piel et al., J Cell Biol., 149:317-330, 2000). The pericentriolar material nucleates the growth of new microtubules; and it serves as a scaffold for molecules that regulate fundamental cellular processes.

### SUMMARY OF THE INVENTION

The compositions and methods described herein are based, at least in part, on the discovery of a centrosome damage checkpoint that can trigger a cell's withdrawal from the cell cycle. The invention provides new methods for treating disorders with a centrosomal etiology, such as carcinoma, tumors, psoriasis, leukemia, Hodgkin's disease, lymphomas, myelofibrosis, and polycythemia vera, and others involving uncontrolled or abnormal patterns of cell division.

Described herein are methods of reducing cell division in a subject by administering to the subject an amount of an inhibitor (e.g., siRNA (e.g., any one of SEQ ID NO:1-26), antibodies (e.g., monoclonal or polyclonal antibodies produced in vivo or in vitro), aptamers, ribozymes, antisense nucleic acids, and small molecules) of centrosomal gene expression effective to decrease cell division (e.g., due to carcinoma, tumors, psoriasis, cancers, leukemia, Hodgkin's disease, lymphomas, myelofibrosis, polycythemia vera, or another cell proliferative disorder) in the subject. Examples of centrosomal genes include genes encoding pericentrin, δ-tubulin, ε-tubulin, γ-tubulin, GCP2, GCP3, GCP5, cNAP1, PCM1, NEK2, centrin2, CDC14A, CDC14B, gapcena, nemo, IKK1, and IKK2. These genes are described more fully below.

New methods are described for treating abnormal cell division in a cell. The methods include administering to the cell an amount of a centrosomal gene or a polypeptide encoded by that gene (e.g., pericentrin, δ-tubulin, ε-tubulin, γ-tubulin, GCP2, GCP3, GCP5, cNAP1, PCM1, NEK2, centrin2, CDC14A, CDC14B, gapcena, nemo, IKK1, IKK2) effective to restore normal cell division in a cell.

Methods for treating a subject are described. These treatments can include administering to a subject a composition that decreases function or expression of a centrosomal gene product (e.g. δ-tubulin, ε-tubulin, γ-tubulin, GCP2, GCP3, GCP5, cNAP1, PCM1, NEK2, centrin2, CDC14A, CDC14B, gapcena, nemo, IKK1, IKK2) in an amount sufficient to decrease cell division in the subject. Compositions that can be used in the new methods include nucleic acids (e.g., siRNAs and anti-sense molecules) that include a any one of the sequences of SEQ ID NOs:2-22 and 24-26. Other compositions that can be used to treat a subject include antibodies. These methods can be used to treat conditions that include a carcinoma, tumor, psoriasis, leukemia, Hodgkin's disease, lymphoma, myelofibrosis, or polycythemia vera.

Also described are screening methods for identifying anti-proliferative compounds, e.g., by (i) selecting a test compound that inhibits activity or localization of a centrosomal gene product (e.g., δ-tubulin, ε-tubulin, γ-tubulin, GCP2, GCP3, GCP5, cNAP1, PCM1, centrin2, CDC14A, CDC14B), (ii) contacting the compound to a cell under conditions which induce the cell to proliferate when the test compound is absent; and (iii) determining if the test compound induces cell cycle arrest. Test compounds that induce cell cycle arrest are anti-proliferative compounds. In some embodiments, the test compound selected for use in the screening methods is selected for its ability to bind to a centrosomal gene or a polypeptide encoded by a centrosomal gene (e.g., δ-tubulin, ε-tubulin, γ-tubulin, GCP2, GCP3, GCP5, cNAP1, PCM1, centrin2, CDC14A, CDC14sB, gapcena, and nemo). Test compounds that can be used include, but are not limited to, small molecules, antibodies, siRNAs, and antisense RNAs.

Also described herein are compositions that inhibit function or expression of a centrosomal gene product, e.g., δ-tubulin, ε tubulin, γ-tubulin, GCP2, GCP3, GCP5, cNAP1, PCM1, centrin2, CDC14A, CDC14B, gapcena, and nemo. These compositions include siRNAs, antisense RNAs, and polypeptide fragments (e.g., a centrosomal targeting domain) of centrosomal proteins.

Also described herein are compositions that inhibit the function, e.g., localization of a centrosomal gene product, e.g., pericentrin, δ-tubulin, ε tubulin, γ-tubulin, GCP2, GCP3, GCP5, cNAP1, PCM1, centrin2, CDC14A, CDC14B, gapcena, and nemo. In one example, a fragment that includes the centrosomal targeting domain of pericentrin can be used to inhibit centrosomal localization of pericentrin in a cell. These inhibitors of pericentrin localization can be contacted to a cell, e.g. in a subject, to inhibit proliferation of the cell.

As used herein, a “centrosomal gene modulator” is any molecule or other composition capable of increasing or decreasing centrosomal gene expression. Such modulators can include, for example, polypeptides, ribozymes, antisense molecules, siRNA molecules, antibodies, and small molecules.

As used herein, an “inhibitor of centrosomal gene expression” is any molecule or composition capable of decreasing expression of a centrosomal gene product, e.g., at the centrosome. Such inhibitors can include, for example, polypeptides, ribozymes, antisense molecules, siRNA molecules, antibodies, and small molecules.

As used herein a “gene product” refers to the mRNA and/or protein encoded by a gene.

The terms “protein” and “polypeptide” include any chain of amino acids, regardless of length or post-translational modification (e.g., glycosylation or phosphorylation). Thus, the term “centrosomal protein,” includes full-length, naturally occurring wild-type proteins, as well as truncated versions of the wild-type protein.

A “subject”, as used herein, refers to a human or a non-human animal, such as a dog, cat, horse, cow, pig, goat, monkey, or bird.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The new methods provide at least one or more of the following advantages. The new methods of modulating cell division can be used to treat disorders associated with cell proliferation defects, and symptoms thereof. The new methods also enable diagnosis of abnormal centrosomal gene expression or activity, thus providing, for example, valuable insight into the etiology of disorders, or symptoms thereof. Furthermore, the diagnosis of such disorders, or symptoms thereof, can now be followed by previously unavailable methods of treatment.

Other features and advantages of the invention will be apparent from the following detailed description, and from the claims.

### DESCRIPTION OF THE DRAWINGS

FIG. 1A is a Western blot image showing siRNA-mediated reduction of expression for the listed gene products.

FIG. 1B is a pair of fluorescent microscopy images showing siRNA-mediated silencing of GCP2.

FIG. 1C is a histogram quantifying the number of cells in which cdc14a expression was reduced by siRNA treatment.

FIG. 1D is a pair of fluorescence microscopy images showing siRNA-mediated reduction of δ-tubulin expression at the centrosome.

FIG. 1E is a series of fluorescence microscopy images showing siRNA-mediated reduction of expression at the centrosome for the indicated proteins.

FIG. 2A is a series of fluorescence microscopy images showing the effects on cell cycle progression of silencing the indicated genes.

FIG. 2B is a is a pair of fluorescence microscopy images showing the effects on cell cycle progression of silencing the indicated genes.

FIG. 2C is a histogram showing the results of CDK2 kinase assay from cells treated with the indicated siRNA.

FIG. 3A is a series of graphs depicting the effects on cell cycle progression via gene silencing of the indicated genes.

FIG. 3B is a series of graphs depicting the time course of G1 arrest via silencing of GCP-2.

FIG. 4 is a histogram quantifying the induction of G1 arrest by silencing of pericentrin relative to control.

FIG. 5 is a fluorescent microscopy image depicting the induction of G1 arrest by expression of a pericentrin fragment that targeted to the centrosome.

FIG. 6A is a series of graphs showing that p53 is required for G1 arrest via GCP-2 siRNA.

FIG. 6B is a series of fluorescence microscopy images showing that showing that p53 is required for G1 arrest via the indicated siRNA.

FIG. 6C is a histogram quantifying the results of experiment depicted in FIG. 8B for cells treated with pericentrin siRNA.

FIG. 6D is a Western blot image showing that nuclear p53 is activated in cells treated with siRNA to pericentrin.

FIG. 6E is a series of fluorescent microscopy images showing that siRNA directed to GCP-2 causes p53 to localize to the nucleus.

FIG. 6F is a series of fluorescent microscopy images showing that siRNA directed to GCP-2 causes activated p53 to localize to the nucleus.

FIG. 7 is a series of graphs showing that siRNA directed to GCP-3 mediates induction of cell cycle arrest in a p38-dependent manner.

FIG. 8 is a series of graphs depicting induction of G1 arrest via gene silencing of PCM1, cNAP1, GCP2, GCP3, and GCP5.

### DETAILED DESCRIPTION

The new methods disclosed herein are based, at least in part, on the discovery of a centrosome damage checkpoint. The damage checkpoint involves an array of centrosomal genes whose disruption induces (i) delays/defects in cytokinesis and (ii) subsequent G1/G0 arrest. This phenotype resembles the effects observed when centrosomes are experimentally removed from vertebrate cells (See, e.g., Hinchcliffe et al., Science, 291:1547-1550, 2001; and Khodjakov and Rieder, J Cell Biol., 153:237-242, 2001). In addition, the new methods are based, at least in part, on the discovery that centrosomal genes are required for normal functioning of the cell cycle, including cell division.

The methods disclosed herein are also based on the discovery of a centrosome damage checkpoint capable of driving cells out of the cell cycle, thereby reducing or stopping cell division. In some methods, an inhibitor of centrosomal gene expression is used to reduce or stop cell division, e.g., to treat a cell proliferative disorder. These methods can minimize harm to subjects because cell division in adults tends to occur disproportionately among unhealthy, abnormally proliferating cells, rather than healthy, normal cells.

In addition, new methods are provided for rescuing cells with abnormal cell division that (i) do not express functional centrosomal gene product or (ii) express too little centrosomal gene product by delivering copies of centrosomal genes or the polypeptide they encode to affected cells or tissues.

Centrosome Damage Checkpoint

Maintenance of somatic cells requires inheritance of a single centrosome comprised of a centriole pair embedded in the surrounding pericentriolar material (PCM). Many tumor cells, on the other hand, possess centrosomes that are aberrant in size, composition, and number, and exhibit genomic instability. In the Examples described herein, an array of siRNA-mediated gene silencing experiments demonstrated that depletion of several centrosome proteins located in various sites within the centrosome have a common phenotype: withdrawal from the cell cycle. Flow cytometry and Ki-67 staining demonstrated that cells arrested in a G1/G0 state. Arrest was dependent on p53, because p53-deficient cell lines and normal cells treated with siRNAs to reduce p53 levels did not arrest following centrosome gene silencing. To identify the signaling pathway upstream of p53 we used inhibitors and siRNA strategies and found that only p38 inhibitors relieved the cell cycle arrest induced by centrosome gene silencing. This demonstrated that p38 MAP kinase activity was upstream of p53, and was required for the centrosome damage-induced arrest.

The activated (phosphorylated) forms of p53 and p38 localized to the centrosome following siRNA silencing of centrosome genes, suggesting that the relevant signaling occurs at this site. The G1 arrest was specific for centrosome proteins as silencing of several non-centrosomal genes had no effect on cell cycle progression. Moreover, G1 arrest was not triggered by defects in mitosis, cytokinesis, microtubule organization, or by DNA damage. The data are consistent with the existence of a centrosome damage checkpoint that monitors compromised centrosomes and responds by cell cycle withdrawal into G1. This may be a widespread cellular strategy for guarding against duplication of defective centrosomes and prevention of deleterious downstream consequences such as spindle defects, aberrant chromosome segregation, and potentially oncogenic characteristics.

The discovery of a centrosome damage checkpoint allows the manipulation of genes involved in this checkpoint. The new methods can selectively direct cells into a specific stage of the cell cycle. In addition, the new methods can be used to cause cells to exit the cell cycle. This allows the treatment of disorders in which it is advantageous to direct certain cells to exit the cell cycle. Such disorders include carcinoma, tumors, psoriasis, leukemia, Hodgkin's disease, lymphomas, myelofibrosis, and polycythemia vera, and others involving uncontrolled or abnormal patterns of cell division. A particular advantage of the new methods is the disproportionate (targeted) effect such treatment has on abnormal or defective cells because these types of cells tend to divide at higher frequencies than cells with normal, healthy patterns of cell division. Consequently, the new methods not only allow effective treatment against abnormally dividing cells, but offer such treatment while simultaneously minimizing negative effects on normal, healthy cells.

Centrosomal Genes

A number of genes have been identified as playing important roles in the centrosomal apparatus of cells. Such genes are hereafter referred to as “centrosomal genes.” Included among these are pericentrin, δ-tubulin, ε-tubulin, γ-tubulin, GCP2, GCP3, GCP5, cNAP1, PCM1, NEK2, centrin2, CDC14A, CDC14B, gapcena, nemo, IKK1, and IKK2.

PCM1 refers to the gene encoding (or gene product thereof) pericentriolar material 1, which, in humans, encodes transcript variants that include the transcript with GenBank Accession No. NM_{—}006197. In some methods the PCM1 refers to an animal homolog of human PCM1.

Pericentrin A refers to the gene (or gene product of) PCNT1, which, in humans, encodes transcript variants that include the transcript with GenBank Accession No. NM_{—}024844. In some methods the pericentrin A refers to an animal homolog of human pericentrin A.

Pericentrin B refers to the gene (or gene product of) PCNT2 which, in humans, encodes transcript variants that include the transcript with GenBank Accession No. NM_{—}006031. In some methods the pericentrin B refers to an animal homolog of human pericentrin B. Pericentrin B is also known as kendrin.

γ-tubulin refers to the gene (or gene product of) TUBG1 which, in humans, encodes transcript variants that include the transcript with GenBank Accession No. NM_{—}001070. In some methods the γ-tubulin refers to an animal homolog of human γ-tubulin.

δ-tubulin refers to the gene (or gene product of) TUBD1 which, in humans, encodes transcript variants that include the transcript with GenBank Accession No. NM_{—}016261. In some methods the δ-tubulin refers to an animal homolog of human γ-tubulin.

ε-tubulin refers to the gene (or gene product of) TUBE1 which, in humans, encodes transcript variants that include the transcript with GenBank Accession No. NM_{—}016262. In some methods the ε-tubulin refers to an animal homolog of human ε-tubulin.

GCP2 refers to the gene encoding (or gene product thereof) γ-tubulin complex 2 (homolog of yeast SPC97p) which, in humans, encodes transcript variants that include the transcript with GenBank Accession No. AF042378. In some methods GCP2refers to an animal homolog of human GCP2.

GCP3 refers to the gene encoding (or gene product thereof) γ-tubulin complex 3 (homolog of yeast SPC98) which, in humans, encodes transcript variants that include the transcript with GenBank Accession No. AF042379. In some methods GCP3 refers to an animal homolog of human GCP3.

GCP5 refers to the gene encoding (or gene product thereof) tubulin gamma complex associated protein 5 (TUBGCP5), which, in humans, encodes transcript variants that include the transcript with GenBank Accession No. NM_{—}052903. In some methods GCP2 refers to an animal homolog of human GCP5.

cNAP 1 refers to the gene encoding (or gene product thereof) centrosomal Nek2-associated protein 1 described in Fry et al., J. Cell Biol., 141:1563-1574, 1998. In some methods cNAP 1 refers to an animal homolog of human cNAP 1.

NEK2 refers to the gene encoding (or gene product thereof) NIMA (never in mitosis gene a)-related kinase 2), which, in humans, encodes transcript variants that include the transcript with GenBank Accession No. NM_{—}002497. In some methods NEK2 refers to an animal homolog of human NEK2.

centrin2 refers to the gene encoding (or gene product thereof) EF-hand protein, 2 (CETN2), which, in humans, encodes transcript variants that include the transcript with GenBank Accession No. NM_{—}004344. In some methods centrin2 refers to an animal homolog of human centrin2.

CDC14A refers to the gene encoding (or gene product thereof) human homolog A of S. cerevisiae CDC14 cell division cycle 14.

CDC14B refers to the gene encoding (or gene product thereof) human homolog B of S. cerevisiae CDC14 cell division cycle 14.

gapcena refers to the gene encoding (or gene product thereof) RAB GTPase activating protein 1 (RABGAP1), which, in humans, encodes a transcript with GenBank Accession No. NM_{—}012197. In some methods gapcena, refers to an animal homolog of human gapcena.

Nemo refers to the gene encoding (or gene product thereof) NF-kappaB essential modulator (NEMO), a member of the IκB kinase (IKK) core complex.

IKK1 refers to the gene encoding (or gene product thereof) IKKα subunit of the IκB kinase (IKK) core complex IkappaB kinase (IKK) core complex.

IKK2 refers to the gene encoding (or gene product thereof) IKKβ subunit of the IκB kinase (IKK) core complex IkappaB kinase (IKK) core complex.

Centriolin refers to the gene encoding (or gene product thereof) centrosomal protein 1 (CEP1), which, in humans, encodes a transcript with GenBank Accession No. NM_{—}007018. In some methods centriolin, refers to an animal homolog of centriolin.

Ninein refers to the gene encoding (or gene product thereof) GSK3B interacting protein, which, in humans, encodes a transcript with GenBank Accession No. NM_{—}182946. In some methods ninein refers to an animal homolog of human ninein.

The normal function of at least some centrosomal genes can be inhibited in such a manner as to cause affected cells to exit the cell cycle. One important consequence of this exit from the cell cycle is cessation of cell division. In disorders involving abnormally high cell division (e.g., carcinoma, tumors, psoriasis, leukemia, Hodgkin's disease, lymphomas, myelofibrosis, polycythemia vera), one or more centrosomal genes can be inhibited to cause cells involved in the disorder to stop dividing. This method of stopping cell division exerts a targeted effect on cells that are dividing at an abnormally high rate and thus can be a powerful therapy for such disorders.

The new methods involve a number of different techniques to inhibit the function of centrosomal genes.

Methods of Use

Cell division, e.g., in abnormal cells, can be reduced or prevented by, for example, blocking the expression of centrosomal genes (e.g., pericentrin, δ-tubulin, ε-tubulin, γ-tubulin, GCP2, GCP3, GCP5, cNAP1, PCM1, NEK2, centrin2, CDC14A, CDC14B, gapcena, nemo, IKK1, IKK2), or by, for example, inhibiting the activity or interfering with the localization of polypeptides encoded by centrosomal genes. Abnormalities in cell division have been linked to numerous debilitating disorders, including cell proliferation disorders, not the least of which are carcinoma, tumors, psoriasis, leukemia, Hodgkin's disease, lymphomas, myelofibrosis, and polycythemia vera. Because of their pivotal role in modulating cell division, centrosomal genes can be used to treat such disorders at the subcellular level of causation. For example, the new methods can be used to treat cancer by modulating centrosomal genes (e.g., via RNAi, siRNA, antisense nucleic acids, or ribozymes) in cancer cells so as to drive these cells into cell cycle arrest. For example, each one of the siRNA molecules of SEQ ID NOs: 1, 3, and 5 can be used effectively to arrest the cell cycle by inhibiting pericentrin, ε-tubulin, and GCP2, respectively. These and other centrosomal genes can be inhibited individually or in any combination.

In a subject with a cell proliferative disorder these molecules can be delivered to cells in the subject to stop cell proliferation, thereby ameliorating the symptoms of the disorder and slowing or stopping its progression. Such molecules can also be delivered to particular tissues most affected by unhealthy cell proliferation, thus even further localizing cell cycle arrest.

Normal centrosomal gene function can be restored to cells that lack adequate endogenous centrosomal gene product expression or activity to sustain a normal cell division. Restoration is performed by administering therapeutically effective amounts of polypeptides corresponding to these centrosomal genes. This provides a method of treatment for debilitating disorders in which cell proliferation is inadequate for healthy bodily functioning (e.g., in healing of wounds or replacement of epithelial cells). Alternatively, gene therapy can be used to administer nucleic acids that encode the desired centrosomal gene products in the affected tissues.

Abnormal centrosomal gene expression or activity can be diagnosed, thus allowing, for example, valuable insight into the etiology of disorders, or symptoms thereof, that have previously defied medically useful explanation. Having linked any such disorder, or symptom thereof, to abnormal centrosomal gene expression or activity, the new methods then provide treatments heretofore unavailable or unknown.

Gene Silencing

Double-stranded nucleic acid molecules can be used to silence or inhibit expression of a centrosomal gene. RNA interference (RNAi) is a mechanism of post-transcriptional gene silencing in which double-stranded RNA (dsRNA) corresponding to a gene (or coding region) of interest is introduced into a cell or an organism, resulting in degradation of the corresponding mRNA. The RNAi effect persists for multiple cell divisions before gene expression is regained. RNAi is therefore an extremely powerful method for making targeted knockouts or “knockdowns” at the RNA level. RNAi has proven successful in human cells, including human embryonic kidney and HeLa cells (see, e.g., Elbashir et al., Nature, 411:494-8, 2001). In one embodiment, gene silencing can be induced in mammalian cells by enforcing endogenous expression of RNA hairpins (see Paddison et al., PNAS, 99:1443-1448, PNAS). In another embodiment, transfection of small (21-23 nt) dsRNA specifically inhibits gene expression (reviewed in Caplen, Trends in Biotechnology, 20:49-51, 2002).

Although applicants do not intend to be bound by theory, RNAi has been reported to work in the following manner. dsRNA corresponding to a portion of a gene to be silenced is introduced into a cell. The dsRNA is digested into siRNAs, or short interfering RNAs, e.g., 21-23 nucleotides in length. The siRNA duplexes bind to a nuclease complex to form what is known as the RNA-induced silencing complex, or RISC. The RISC targets the homologous transcript by base pairing interactions between one of the siRNA strands and endogenous mRNA. It then cleaves the mRNA ˜12 nucleotides from the 3′ terminus of the siRNA (reviewed in Sharp et al., Genes Dev, 15:485-490, 2001; Hammond et al., Nature Rev Gen, 2:110-119, 2001).

To induce gene silencing, molecular biology techniques can be used to produce dsRNA corresponding to the sequence from a target gene. For example, T7 RNA polymerase can be used to drive simultaneous transcription of both strands of a template DNA (corresponding to the target sequence). Kits for production of dsRNA for use in RNAi are available commercially (e.g., from New England Biolabs, Inc. (Beverly, Mass.) or Ambion, Inc., (Austin, Tex.)). Plasmids engineered to make dsRNA, or dsRNA itself can be transfected into cells.

Gene silencing effects similar to those of RNAi have been reported in mammalian cells with transfection of a mRNA-cDNA hybrid construct (Lin et al., Biochem. Biophys. Res. Commun., 281(3):639-44, 2001), providing yet another strategy for gene silencing.

siRNA sequences to centrosomal genes can be designed, for example, using siDESIGN Center (e.g., found at dharmacon.com on the internet) or the Custom SMARTPool® siRNA Design Service (Dharmacon Research, Inc.). Given a particular nucleic acid sequence, one can generate numerous siRNA molecules that can silence genes. The invention contemplates the generation (e.g., by these or other methods of producing siRNA molecules) and use of myriad siRNA molecules capable of silencing centrosomal genes. Representative examples of such siRNA molecules are provided herein.

Examples of siRNA sequences (along with the target centrosomal gene to be inhibited) that can be used in the new methods include the following (all listed from 5′ to 3′ (sense) AA-N19-dTdT:

| | |
|---|---|
| pericentrin A/B | GCAGCUGAGCUGAAGGAGA (SEQ ID NO:1) |
| pericentrin B | UUGGAACAGCUGCAGCAGA (SEQ ID NO:23) |
| δ-tubulin | GGUUCUGGAAACAACUGGG (SEQ ID NO:2) |
| ε-tubulin | AGUCGGCAGAGCACUGUGA (SEQ ID NO:3) |
| γ-tubulin | UGACCGCAAGGACGUCUUU (SEQ ID NO:4) |
| GCP2 | UCUCGUACUCCAGAAGACU (SEQ ID NO:5) |
| GCP3 | AAGAGAAGCAGAUGCUGCA (SEQ ID NO:6) |
| GCP5 | ACUUCGCCUGGUCCAACUU (SEQ ID NO:7) |
| cNAP1 | UCUAUCCGAAAGCCCAGUC (SEQ ID NO:8) |
| PCM1 | GUCCCCCAAACAGAGAAAC (SEQ ID NO:9) |
| PCM1 (alternate) | UCAGCUUCGUGAUUCUCAG (SEQ ID NO:10) |
| NEK2 | AGGGAACCAAGGAAAGGCA (SEQ ID NO:11) |
| centrin2 | GAGCAAAAGCAGGAGAUCC (SEQ ID NO:12) |
| CDC14A | GCACAGUAAAUACCCACUA (SEQ ID NO:13) |
| CDC14B | GCAAAUGCUGCCUUCCUUG (SEQ ID NO:14) |
| gapcena | CCAGAGAUGAGCCUACCAG (SEQ ID NO:15) |
| nemo | GAGAUGCCAGCAGCAGAUG (SEQ ID NO:16) |
| IKK1 | ACAGAGAACGAUGGUGCCA (SEQ ID NO:17) |
| IKK2 | UCAGGAAACAGGUGAGCAG (SEQ ID NO:18) |
| Centriolin | GGAUCAGAGACUCUACCUU (SEQ ID NO:19) |
| Ninein | GUGCUGCAGCAGACAUUAC (SEQ ID NO:20) |
| Ninein | UAUGAGCAUUGAGGCAGAG (SEQ ID NO:21) |
| (alternate) | |
| CNAP1 | CUGUCACUCAAGCCAAGGA (SEQ ID NO:22) |
| (alternate) | |

In this list of exemplary siRNA sequences: pericentrin A/B siRNA is directed to both pericentrin A and pericentrin B, while pericentrin B siRNA is directed only to pericentrin B (i.e., Kendrin). Sequences designated as “alternate” represent a second exemplary siRNA that can be used to silence the indicated genes in one or more of the methods described herein.

These are merely included as a representative sample of the many centrosomal gene siRNA sequences that can be employed in the invention. Other siRNA Molecules include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules of the invention can be chemically synthesized, or can transcribed be in vitro from a DNA template, or in vivo from, e.g., shRNA. The dsRNA molecules can be designed using any method known in the art. A number of algorithms are known in the art, and include the following general protocol:

1. Beginning with the AUG start codon, look for AA dinucleotide sequences; each AA and the 3′ adjacent 16 or more nucleotides are potential siRNA targets. siRNAs taken from the 5′ untranslated regions (UTRs) and regions near the start codon (within about 75 bases or so) may be less useful as they may be richer in regulatory protein binding sites, and UTR-binding proteins and/or translation initiation complexes may interfere with binding of the siRNP or RISC endonuclease complex. Thus, in one embodiment, the nucleic acid molecules are selected from a region of the cDNA sequence beginning 50 to 100 nt downstream of the start codon. Further, siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules can have a 3′ overhang of 2 nucleotides, such as TT. The overhanging nucleotides can be either RNA or DNA.

2. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at www.ncbi.nlm.nih.gov/BLAST.

3. Select one or more sequences that meet your criteria for evaluation.

Further information about the design and use of siRNA can be found in “The siRNA User Guide,” available at www.mpibpc.gwdg.de/abteilungen/100/105/sirna.html. Alternatively, siRNAs or pools thereof can be obtained from commercial vendors including Ambion and Dharmacon.

Negative control siRNAs can have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The nucleic acids described herein include both siRNA and crosslinked siRNA derivatives. Crosslinking can be employed to alter the pharmacokinetics of the composition, for example, to increase half-life in the body. Thus, siRNA derivatives can include siRNA having two complementary strands of nucleic acid, such that the two strands are crosslinked. For example, a 3′ OH terminus of one of the strands can be modified, or the two strands can be crosslinked and modified at the 3′OH terminus. The siRNA derivative can contain a single crosslink (e.g., a psoralen crosslink). In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

siRNAs can be unconjugated or can be conjugated to another moiety, such as a nanoparticle, to enhance a pharmacokinetic parameter such as absorption, efficacy, bioavailability, and/or half-life. The conjugation can be accomplished by methods known in the art, e.g., using the methods of Lambert et al., Drug Deliv. Rev.:47(1), 99-112 (2001) (describes nucleic acids loaded to polyalkylcyanoacrylate (PACA) nanoparticles); Fattal et al., J. Control Release, 53:137-43, 1998 (describes nucleic acids bound to nanoparticles); Schwab et al., Ann. Oncol., 5 Suppl. 4:55-8, 1994 (describes nucleic acids linked to intercalating agents, hydrophobic groups, polycations or PACA nanoparticles); and Godard et al., Eur. J. Biochem., 232:404-10, 1995 (describes nucleic acids linked to nanoparticles).

siRNA molecules of the present invention can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P, or other appropriate isotope.

Synthetic siRNAs can be delivered into cells by cationic liposome transfection and electroporation. These exogenous siRNA sometimes exert only a short term silencing effect (4˜5 days). Several strategies for expressing siRNA duplexes within cells from recombinant DNA constructs allow longer-term target gene suppression in cells, including mammalian Pol III promoter systems (e.g., H1 or U6/snRNA promoter systems capable of expressing functional double-stranded siRNAs; (See, e.g., Bagella et al., J. Cell. Physiol. 177:206-213, 1998). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al., 1998), supra). Constructs containing siRNA sequence under the control of T7 promoter also make functional siRNAs when cotransfected into the cells with a vector expression T7 RNA polymerase.

Animal cells express a range of noncoding RNAs of approximately 22 nucleotides termed micro RNA (miRNAs) and can regulate gene expression at the post transcriptional or translational level during animal development. One common feature of miRNAs is that they are all excised from an approximately 70 nucleotide precursor RNA stem-loop, probably by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells. When expressed by DNA vectors containing polymerase III promoters, micro-RNA designed hairpins can silence gene expression. Viral-mediated delivery mechanisms can also be used to induce specific silencing of targeted genes through expression of siRNA, for example, by generating recombinant adenoviruses harboring siRNA under RNA Pol II promoter transcription control. Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al., Proc. Natl. Acad. Sci. USA, 99:14236-40, 2002). In adult mice, efficient delivery of siRNA can be accomplished by “high-pressure” delivery technique, a rapid injection (within 5 seconds) of a large volume of siRNA containing solution into animal via the tail vein (See, e.g., Lewis, Nature Genetics, 32:107-108, 2002). Nanoparticles and liposomes can also be used to deliver siRNA into animals.

Engineered RNA precursors, introduced into cells or whole organisms as described herein, will lead to the production of a desired siRNA molecule. Such an siRNA molecule will then associate with endogenous protein components of the RNAi pathway to bind to and target a specific mRNA sequence for cleavage and destruction. In this fashion, the mRNA to be targeted by the siRNA generated from the engineered RNA precursor will be depleted from the cell or organism, leading to a decrease in the concentration of the protein encoded by that mRNA in the cell or organism.

Polypeptides

Polypeptides that are fragments of proteins encoded by centrosomal genes can be used to disrupt the function of endogenous centrosomal genes. Some useful polypeptides include at least part of a functional domain from a centrosomal protein. For example, polypeptides can include a binding domain that mediates protein-protein interactions between a centrosomal protein and a second protein. When introduced in sufficient quantity to a cell, such a polypeptide can compete with an endogenous centrosomal protein for binding with the second protein, thereby disrupting the ability of the centrosomal protein to function via competitive binding inhibition, and thereby inhibit proliferation of the cell. In many cases, the fragment outcompetes the endogenous protein and exerts a dominant negative phenotype. Methods for introducing a polypeptide are provided below.

In other examples, useful polypeptides can contain the centrosomal localization signal of a centrosomal protein. When introduced in sufficient quantity to a cell, such a peptide or fragment can saturate the machinery and/or the ligand(s) responsible for localizing endogenous centrosomal protein to the centrosome, thereby preventing the centrosomal protein from localizing to and/or functioning at the centrosome. An example of a fragment that disrupts localization of pericentrin is described in Example 5, infra.

In a different aspect, for some subjects it will be useful to restore or introduce the centrosomal protein function in a cell to induce cell proliferation. In these methods useful polypeptides include full-length wild-type centrosomal proteins and/or fragments and variants thereof that retain the centrosomal function of wild type protein.

Polypeptides can be introduced to cells in a number ways. For example, polypeptides can by formulated into pharmaceutical compositions suitable for topical administration. Polypeptides can be formulated in a cell penetrant such as DMSO. Polypeptides can be delivered by transfection using a liposome carrier or, in some cases, by electroporation. Methods for the sustained delivery of polypeptides include introduction of a recombinant nucleic acid designed to express the polypeptide in the cell. Such nucleic acids can include plasmid or viral vectors that include a promoter suitable for driving expression of a sequence encoding the polypeptide. In other methods, Nucleic acids can include promoters that

Antibodies

Full length proteins or polypeptides of centrosomal genes, or immunogenic fragments or analogs thereof, can be used to raise antibodies useful in the methods described herein; such polypeptides can be produced by recombinant techniques or synthesized (see, for example, “Solid Phase Peptide Synthesis,” supra; Sambrook et al., Molecular Cloning: A Laboratory Manual, Cold Spring Laboratory, Cold Spring Harbor, N.Y. (1989); Ausubel et al. (Eds.), Current Protocols in -Molecular Biology, John Wiley & Sons, New York, N.Y., 1999 and preceding editions; and U.S. Pat. No. 4,237,224). In general, the peptides can be coupled to a carrier protein, such as KLH, as described in Ausubel et al., supra, mixed with an adjuvant, and injected into a host mammal. Antibodies can be purified by peptide antigen affinity chromatography.

In particular, various host animals can be immunized by injection with a polypeptide of the invention. Host animals include rabbits, mice, guinea pigs, and rats. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum. Polyclonal antibodies are heterogeneous populations of antibody molecules that are contained in the sera of the immunized animals.

Antibodies for use in the new methods include polyclonal antibodies and, in addition, monoclonal antibodies, humanized or chimeric antibodies, single chain antibodies, Fab fragments, F(ab′)₂ fragments, and molecules produced using a Fab expression library.

Monoclonal antibodies (mAbs), which are homogeneous populations of antibodies to a particular antigen, can be prepared using the polypeptides of the invention described above and standard hybridoma technology (see, for example, Kohler et al., Nature, 256:495, 1975; Kohler et al., Eur. J. Immunol., 6:511, 1976; Kohler et al., Eur. J. Immunol, 6:292, 1976; Hammerling et al., “Monoclonal Antibodies and T Cell Hybridomas,” Elsevier, N.Y., 1981; Ausubel et al., supra).

In particular, monoclonal antibodies can be obtained by any technique that provides for the production of antibody molecules by continuous cell lines in culture such as described in Kohler et al. (Nature, 256:495, 1975, and U.S. Pat. No. 4,376,110); the human B-cell hybridoma technique (Kosbor et al., Immunology Today, 4:72, 1983; Cole et al., Proc. Natl. Acad. Sci. USA, 80:2026, 1983); and the EBV-hybridoma technique (Cole et al., “Monoclonal Antibodies and Cancer Therapy,” Alan R. Liss, Inc., pp. 77-96, 1983). Such antibodies can be of any immunoglobulin class including IgG, IgM, IgE, IgA, IgD and any subclass thereof. Hybridomas producing mAbs may be cultivated in vitro or in vivo. The ability to produce high titers of mAbs in vivo makes this a particularly useful method of production.

Once produced, polyclonal or monoclonal antibodies are tested for specific recognition of centrosomal proteins, or fragment thereof, by Western blot or immunoprecipitation analysis by standard methods (e.g., as described in Ausubel et al., supra). Antibodies that specifically recognize and bind to centrosomal proteins, or fragments thereof, are useful in the methods described herein. For example, such antibodies can be used in an immunoassay to monitor a centrosomal protein, or fragment thereof, in mammalian cells (for example, to determine the amount or subcellular location of a centrosomal protein, or fragment thereof).

Preferably, antibodies are produced using fragments of the protein of the invention that lie outside highly conserved regions and appear likely to be antigenic, by criteria such as high frequency of charged residues. In one specific example, such fragments are generated by standard techniques of PCR, and are then cloned into the pGEX expression vector (Ausubel et al., supra). Fusion proteins are expressed in E. coli and purified using a glutathione agarose affinity matrix as described in Ausubel, et al., supra.

In some cases it may be desirable to minimize the potential problems of low affinity or specificity of antisera. In such circumstances, two or three fusions can be generated for each protein, and each fusion can be injected into at least two rabbits. Antisera can be raised by injections in a series, preferably including at least three booster injections.

Antisera may also be checked for its ability to immunoprecipitate recombinant proteins of the invention or control proteins, such as glucocorticoid receptor, CAT, or luciferase.

In other embodiments, techniques developed for the production of “chimeric antibodies” (Morrison et al., Proc. Natl. Acad. Sci. USA, 81:6851, 1984; Neuberger et al., Nature, 312:604, 1984; Takeda et al., Nature, 314:452, 1984) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Generally, partially human antibodies and fully human antibodies have a longer half-life within the human body than other antibodies. Accordingly, lower dosages and less frequent administration are often possible. Modifications such as lipidation can be used to stabilize antibodies and to enhance uptake and tissue penetration (e.g., into the brain). A method for lipidation of antibodies is described by Cruikshank et al. (J. Acquired Immune Deficiency Syndromes and Human Retrovirology, 14:193, 1997).

Alternatively, techniques described for the production of single chain antibodies (U.S. Pat. Nos. 4,946,778, 4,946,778, and 4,704,692) can be adapted to produce single chain antibodies against centrosomal proteins, or fragments thereof. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments include but are not limited to F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed (Huse et al., Science, 246:1275, 1989) to allow rapid and easy identification of monoclonal Fab fragments with the desired specificity.

Antibodies to centrosomal polypeptides can, in turn, be used to generate anti-idiotype antibodies using techniques well known to those skilled in the art (see, e.g., Greenspan et al., FASEB J., 7:437, 1993; Nissinoff, J. Immunol., 147:2429, 1991). For example, antibodies that bind to a centrosomal polypeptide and competitively inhibit the binding of a binding partner of that centrosomal polypeptide can be used to generate anti-idiotypes that resemble a binding partner binding domain of the protein and, therefore, bind and neutralize a binding partner of the protein. Such neutralizing anti-idiotypic antibodies or Fab fragments of such anti-idiotypic antibodies can be used in therapeutic regimens.

Antibodies can be humanized by methods known in the art. For example, monoclonal antibodies with a desired binding specificity can be commercially humanized (Scotgene, Scotland; Oxford Molecular, Palo Alto, Calif.). Fully human antibodies, such as those expressed in transgenic animals are also features of the invention (Green et al., Nature Genetics, 7:13-21, 1994; see also U.S. Pat. Nos. 5,545,806 and 5,569,825).

Antibodies to centrosomal proteins can be used in therapeutic uses described herein, e.g., by inhibiting function of centrosomal gene products and thereby arresting cell-cycle progression. Antibodies can be used, for example, in the detection of centrosomal polypeptides of the invention in a biological sample as part of a diagnostic assay. Antibodies also can be used in a screening assay to measure the effect of a candidate compound on expression or localization of a centrosomal protein, or fragment thereof. Additionally, such antibodies can be used in conjunction with gene therapy techniques to, for example, evaluate normal and/or genetically engineered cells that express nucleic acids or polypeptides described herein prior to their introduction into a subject. Such antibodies additionally can be used in methods for inhibiting abnormal activity of nucleic acids or polypeptides described herein.

The methods described herein in which antibodies to centrosomal polypeptides are employed may be performed, for example, by utilizing pre-packaged diagnostic kits comprising at least one such specific antibody described herein, which may be conveniently used, for example, in clinical settings, to diagnose subjects exhibiting symptoms of disorders associated with aberrant expression of nucleic acids or polypeptides of the invention.

Conjugated antibodies (i.e., antibodies joined to a moiety of a drug molecule) can be used for modifying a given biological response. The conjugated drug moiety need not be limited to classical chemical therapeutic agents. For example, the drug moiety can be a protein or polypeptide possessing a desired biological activity. Such proteins include, for example, toxins such as abrin, ricin A, Pseudomonas exotoxin, or Diphtheria toxin; proteins such as tumor necrosis factor, alpha-interferon, beta-interferon, nerve growth factor, platelet derived growth factor, tissue plasminogen activator; and biological response modifiers such as lymphokines, interleukin-1, interleukin-2, interleukin-6, granulocyte macrophage colony stimulating factor, granulocyte colony stimulating factor, or other growth factors.

Techniques for conjugating a therapeutic moiety to an antibody are well known (see, e.g., Arnon et al., 1985, “Monoclonal Antibodies For Immunotargeting Of Drugs In Cancer Therapy”, in Monoclonal Antibodies And Cancer Therapy, Reisfeld et al., Eds., Alan R. Liss, Inc. pp. 243-256; Hellstrom et al., 1987, “Antibodies For Drug Delivery”, in Controlled Drug Delivery, 2nd ed., Robinson et al., Eds., Marcel Dekker, Inc., pp. 623-653; Thorpe, 1985, “Antibody Carriers Of Cytotoxic Agents In Cancer Therapy: A Review”, in Monoclonal Antibodies '84: Biological And Clinical Applications, Pinchera et al., Eds., pp. 475-506; “Analysis, Results, And Future Prospective Of The Therapeutic Use Of Radiolabeled Antibody In Cancer Therapy”, in Monoclonal Antibodies For Cancer Detection And Therapy, Baldwin et al., Eds., Academic Press, pp. 303-316, 1985; and Thorpe et al., 1982, Immunol. Rev., 62:119-158). Alternatively, an antibody can be conjugated to a second antibody to form an antibody heteroconjugate as described by Segal in U.S. Pat. No. 4,676,980.

Aptamers

Aptamers are nucleic acid molecules that bind to specific target molecules based on their three-dimensional conformation rather than hybridization. The aptamers are selected, for example, by synthesizing an initial heterogeneous population of oligonucleotides, and then selecting oligonucleotides within the population that bind tightly to a particular target molecule. Once an aptamer that binds to a particular target molecule has been identified, it can be replicated using a variety of techniques known in biological and other arts, e.g., by cloning and polymerase chain reaction (PCR) amplification followed by transcription. The target molecules can be nucleic acids, proteins, peptides, small organic and inorganic compounds, and even entire micro-organisms.

The synthesis of a heterogeneous population of oligonucleotides and the selection of aptamers within that population can be accomplished using a procedure known as the Systematic Evolution of Ligands by Exponential Enrichment or SELEX. The SELEX method is described in, e.g., Gold et al., U.S. Pat. Nos. 5,270,163 and 5,567,588; Fitzwater et al., (Methods in Enzymology, 267:275-301, 1996); and in Ellington and Szostak (Nature, 346:818-22). Briefly, a heterogeneous DNA oligomer population is synthesized to provide candidate oligomers for the in vitro selection of aptamers. This initial DNA oligomer population is a set of random sequences 15 to 100 nucleotides in length flanked by fixed 5′ and 3′ sequences 10 to 50 nucleotides in length. The fixed regions provide sites for PCR primer hybridization and, in one implementation, for initiation of transcription by an RNA polymerase to produce a population of RNA oligomers. The fixed regions also contain restriction sites for cloning selected aptamers. Many examples of fixed regions can be used in aptamer evolution (see, e.g., Conrad et al., Methods in Enzymology, 267:336-83, 1996; Ciesiolka et al., Methods in Enzymology, 267:315-35, 1996; Fitzwater, supra).

Aptamers are selected in a 5 to 100 cycle procedure. In each cycle, oligomers are bound to the target molecule, purified by isolating the target to which they are bound, released from the target, and then replicated by 20 to 30 generations of PCR amplification.

Aptamer selection is similar to evolutionary selection of a function in biology. Subjecting the heterogeneous oligonucleotide population to the aptamer selection procedure described above is analogous to subjecting a continuously reproducing biological population to 10 to 20 severe selection events for the function, with each selection separated by 20 to 30 generations of replication.

Heterogeneity is introduced, e.g., only at the beginning of the aptamer selection procedure, and does not occur throughout the replication process. Alternatively, heterogeneity can be introduced at later stages of the aptamer selection procedure.

Various oligomers can be used for aptamer selection, including, e.g., 2′-fluoro-ribonucleotide oligomers, NH2-substituted and OCH3-substituted ribose aptamers, and deoxyribose aptamers. RNA and DNA populations are equally capable of providing aptamers configured to bind to any type of target molecule. Within either population, the selected aptamers occur at a frequency of 109 to 1013 (e.g., Gold et al., Annual Review of Biochemistry, 64:763-97, 1995), and most frequently have nanomolar binding affinities to the target, affinities as strong as those of antibodies to cognate antigens (e.g., Griffiths et al, EMBO J., 13:3245-60, 1994).

Using 2′-fluoro-ribonucleotide oligomers is likely to increase binding affinities ten to one hundred fold over those obtained with unsubstituted ribo- or deoxyribo-oligonucleotides (e.g., Pagratis et al., Nature Biotechnology, 15:68-73). Such modified bases provide additional binding interactions and increase the stability of aptamer secondary structures. These modifications also make the aptamers resistant to nucleases, a significant advantage for real world applications of the system (e.g., Lin et al., Nucleic Acids Research, 22:5229-34, 1994; Pagratis, supra).

In the present invention, aptamers can be made that specifically bind to centrosomal nucleic acids, or to polypeptides they encode, using the techniques described herein.

Antisense Nucleic Acids

Treatment regimes based on an “antisense” approach involve the design of oligonucleotides (either DNA or RNA) that are complementary to mRNA complementary to centrosomal genes (e.g., pericentrin, δ-tubulin, ε-tubulin, γ-tubulin, GCP2, GCP3, GCP5, cNAP1, PCM1, NEK2, centrin2, CDC14A, CDC14B, gapcena, nemo, IKK1, and IKK2). These oligonucleotides bind to the complementary mRNA transcripts of centrosomal genes and prevent translation. Absolute complementarity, although preferred, is not required. A sequence “complementary” to a portion of an RNA, as referred to herein, means a sequence having sufficient complementarily to be able to hybridize with the RNA in vivo, forming a stable duplex; in the case of double-stranded antisense nucleic acids, a single strand of the duplex DNA may be tested, or triplex formation may be assayed. The ability to hybridize will depend on both the degree of complementarily and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, for example, the 5′ untranslated sequence up to and including the AUG initiation codon, should work efficiently at inhibiting translation. However, sequences complementary to the 3′ untranslated sequences of mRNAs recently have been shown to be effective at inhibiting translation of mRNAs as well (Wagner, Nature, 372:333, 1984). Thus, oligonucleotides complementary to either the 5′ or 3′ non-translated, non-coding regions of the gene or mRNA could be used in an antisense approach to inhibit translation of endogenous mRNA. Oligonucleotides complementary to the 5′ untranslated region of the mRNA should include the complement of the AUG start codon.

Antisense oligonucleotides complementary to mRNA coding regions are less efficient inhibitors of translation, but could be used in accordance with the invention. Whether designed to hybridize to the 5′, 3′, or coding region of an mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides, or at least 50 nucleotides. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

Based upon the sequences of centrosomal genes, one of skill in the art can choose and synthesize any of a number of appropriate antisense molecules for use in accordance with the present invention. For example, a “gene walk” comprising a series of oligonucleotides of 15-30 nucleotides spanning the length of a centrosomal gene nucleic acid can be prepared, followed by testing for inhibition of centrosomal gene expression. Optionally, gaps of 5-10 nucleotides can be left between the oligonucleotides to reduce the number of oligonucleotides synthesized and tested.

Regardless of the choice of target sequence, it is preferred that in vitro studies are first performed to quantitate the ability of the antisense oligonucleotide to inhibit gene expression. These studies can utilize controls that distinguish between antisense gene inhibition and nonspecific biological effects of oligonucleotides. These studies can also compare levels of the target RNA or protein with that of an internal control RNA or protein. Additionally, it is envisioned that results obtained using the antisense oligonucleotide are compared with those obtained using a control oligonucleotide. The control oligonucleotide can be of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, and can be single-stranded or double-stranded. The oligonucleotides can be modified at the base moiety, sugar moiety, or phosphate backbone, for example, to improve stability of the molecule, hybridization, etc. The oligonucleotides may include other appended groups such as peptides (e.g., for targeting host cell receptors in vivo), or agents facilitating transport across the cell membrane (as described, e.g., in Letsinger et al., Proc. Natl. Acad. Sci. USA, 86:6553, 1989; Lemaitre et al., Proc. Natl. Acad. Sci. USA, 84:648, 1987; PCT Publication No. WO 88/09810) or the blood-brain barrier (see, for example, PCT Publication No. WO 89/10134), or hybridization-triggered cleavage agents (see, for example, Krol et al., BioTechniques, 6:958, 1988), or intercalating agents (see, for example, Zon, Pharm. Res., 5:539, 1988). To this end, the oligonucleotides can be conjugated to another molecule, for example, a peptide, hybridization triggered cross-linking agent, transport agent, or hybridization-triggered cleavage agent.

The antisense oligonucleotides can include at least one modified base moiety which is selected from the group including, but not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethyl-aminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6isopentenyladenine, uracil-5-oxyacetic acid (v), wybutoxosine, pseudouracil, queosine, 2-thiocytosine, 5-methyl-2-theouracil, 2-thiouracil, 4-thiouracil, 5-methyluracil, uracil-5-oxyacetic acid methylester, uracil-5-oxyacetic acid (v), 5-methyl-2-thiouracil, 2-(3-amino-3-N-2-carboxypropl) uracil, (acp3)w, and 2,6-diaminopurine.

The antisense oligonucleotides can also comprise at least one modified sugar moiety selected from the group including, but not limited to, arabinose, 2-fluoroarabinose, xylulose, and hexose.

In yet another embodiment, the antisense oligonucleotides include at least one modified phosphate backbone selected from the group of a phosphorothioate, a phosphorodithioate, a phosphoramidothioate, a phosphoramidate, a phosphordiamidate, a methylphosphonate, an alkyl phosphotriester, and a formacetal, or an analog of any of these backbones.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids. Res., 15:6625, 1987). The oligonucleotide is a 2′-0-methylribonucleotide (Inoue et al., Nucl. Acids Res., 15:6131, 1987), or a chimeric RNA-DNA analog (Inoue et al., FEBS Lett., 215:327, 1987).

Then new antisense oligonucleotides can be synthesized by standard methods known in the art, e.g., by use of an automated DNA synthesizer (such as are commercially available from Biosearch, Applied Biosystems, etc.). As examples, phosphorothioate oligonucleotides can be synthesized by the method of Stein et al. (Nucl. Acids Res., 16:3209, 1988), and methylphosphonate oligonucleotides can be prepared by use of controlled pore glass polymer supports (Sarin et al., Proc. Natl. Acad. Sci. USA, 85:7448, 1988). Examples of antisense molecules that can be used in the methods of the invention include RNA molecules identical to the sequences stretching from nucleotide positions 81-183, 245-356, and 409-525 of SEQ ID NO:1 or positions 167-334, 346-478, and 534-656 of SEQ ID NO:3, with U's substituted for all T's. Many other such examples of antisense molecules are also available.

Antisense molecules can be delivered to cells that express nucleic acids or polypeptides in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense molecule sufficient to suppress translation of endogenous mRNAs. Therefore, a preferred approach uses a recombinant DNA construct in which the antisense oligonucleotide is placed under the control of a strong pol III or pol II promoter. The use of such a construct to transfect target cells in the subject will result in the transcription of sufficient amounts of single stranded RNAs that will form complementary base pairs with the endogenous transcripts of nucleic acids of the invention and thereby prevent translation of the endogenous mRNA. The invention encompasses the construction of an antisense RNA using the complementary strand as a template. For example, a vector can be introduced in vivo such that it is taken up by a cell and directs the transcription of an antisense RNA. Such a vector can remain episomal or become chromosomally integrated, as long as it can be transcribed to produce the desired antisense RNA.

Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include, but are not limited to: the SV40 early promoter region (Bernoist et al., Nature 290:304, 1981); the promoter contained in the 3′ long terminal repeat of Rous sarcoma virus (Yamamoto et al., Cell, 22:787-797, 1988); the herpes thymidine kinase promoter (Wagner et al., Proc. Natl. Acad. Sci. USA, 78:1441, 1981); or the regulatory sequences of the metallothionein gene (Brinster et al., Nature, 296:39, 1988).

Ribozymes

Ribozyme molecules designed to catalytically cleave mRNA transcripts of centrosomal genes (e.g., pericentrin, δ-tubulin, ε-tubulin, γ-tubulin, GCP2, GCP3, GCP5, cNAP1, PCM1, NEK2, centrin2, CDC14A, CDC14B, gapcena, nemo, IKK1, and IKK2) can be used to prevent translation and expression of centrosomal genes. (e.g., PCT Publication WO 90/11364; Saraver et al., Science, 247:1222, 1990). While various ribozymes that cleave mRNA at site-specific recognition sequences can be used to destroy centrosomal gene mRNAs, the use of hammerhead ribozymes can be very effective. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art (Haseloffet al., Nature, 334:585, 1988). Preferably, the ribozyme is engineered so that the cleavage recognition site is located near the 5′ end of the mRNA, that is, to increase efficiency and minimize the intracellular accumulation of non-functional mRNA transcripts.

The new ribozymes also include RNA endoribonucleases (hereinafter “Cech-type ribozymes”), such as the one that occurs naturally in Tetrahymena thermophila (known as the IVS or L-19 IVS RNA), and which has been extensively described by Cech and his collaborators (Zaug et al., Science, 224:574, 1984; Zaug et al., Science, 231:470, 1986; Zug et al., Nature, 324:429, 1986; PCT Application No. WO 88/04300; and Been et al., Cell, 47:207, 1986). The Cech-type ribozymes have an eight base-pair sequence that hybridizes to a target RNA sequence, whereafter cleavage of the target RNA takes place. The invention encompasses those Cech-type ribozymes that target eight base-pair active site sequences present in nucleic acids of the invention.

As in the antisense approach, the ribozymes can be composed of modified oligonucleotides (e.g., for improved stability, targeting), and should be delivered to cells that express nucleic acids or polypeptides of the invention in vivo. A preferred method of delivery involves using a DNA construct “encoding” the ribozyme under the control of a strong constitutive pol III or pol II promoter, so that transfected cells will produce sufficient quantities of the ribozyme to destroy endogenous messages and inhibit translation. Because ribozymes, unlike antisense molecules, are catalytic, a lower intracellular concentration is required for efficiency.

Gene Therapy

Nucleic acid molecules encoding siRNA molecules, antisense molecules, polypeptides, and antibodies described herein can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In some examples, subjects suffering from a cell proliferative disorder can be treated with a gene therapy vector encoding and siRNA molecules, antisense molecules, polypeptide fragment, or antibody that inhibits centrosomal gene product function and thereby induces cell cycle arrest in proliferative cells.

In other examples, a gene therapy vector encoding a centrosomal protein, or functional fragment thereof, can be delivered to a subject suffering from a disorder attended by insufficient cell proliferation. For example, a gene therapy vector encoding pericentrin, δ-tubulin, ε-tubulin, γ-tubulin, GCP2, GCP3, GCP5, cNAP1, PCM1, NEK2, centrin2, CDC14A, CDC14B, gapcena, nemo, IKK1, IKK2 is delivered to a subject suffering from a wound to thereby stimulate proliferation of cells involved in wound healing, e.g., repair tissue.

Screening Assays

Compounds with unknown function can be screened to determine whether they specifically bind to centrosomal genes or the polypeptides they encode using any standard binding assay. For example, candidate compounds can be bound to a solid support. A centrosomal gene or polypeptide is then exposed to the immobilized compound and binding is measured (e.g., as done in European Patent Application 84/03564). Methods of measuring binding include, for example, enzyme-linked immunosorbent assay (ELISA). ELISAs can be performed using high-throughput methods.

In one embodiment, the invention provides assays for screening candidate or test compounds that bind to or modulate the activity of a polypeptide encoded by a centrosomal gene, or biologically active portion of that polypeptide. The test, or “candidate”, compounds can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer, or small molecule libraries of compounds (Lam, Anticancer Drug Des., 12:145, 1997).

Examples of methods useful for the synthesis of molecular libraries can be found in the art (e.g., DeWitt et al., Proc. Natl. Acad. Sci. USA, 90:6909, 1993; Erb et al., Proc. Natl. Acad. Sci. USA, 91:11422, 1994; Zuckermann et al., J. Med. Chem., 37:2678, 1994; Cho et al., Science, 261:1303, 1993; Carrell et al., Angew. Chem. Int. Ed. Engl., 33:2059, 1994; Carell et al., Angew. Chem. Int. Ed. Engl., 33:2061, 1994; and Gallop et al., J. Med. Chem., 37:1233. 1994).

Libraries of compounds can be presented in solution (e.g., Houghten, Bio/Techniques, 13:412-421, 1992), or on beads (Lam, Nature, 354:82-84, 1991), chips (Fodor, Nature, 364:555-556, 1993), bacteria (U.S. Pat. No. 5,223,409), spores (U.S. Pat. Nos. 5,571,698; 5,403,484; and 5,223,409), plasmids (Cull et al., Proc. Natl. Acad. Sci. USA, 89:1865-1869, 1992) or phage (Scott and Smith, Science, 249:386-390, 1990; Devlin, Science, 249:404-406, 1990; Cwirla et al., Proc. Natl. Acad. Sci. USA, 87:6378-6382, 1990; and Felici, J. Mol. Biol., 222:301-310, 1991).

In one embodiment, an assay is a cell-based assay in which a cell that expresses a membrane-bound form of a centrosomal polypeptide, or a biologically active portion thereof, on the cell surface is contacted with a test compound and the ability of the test compound to bind with the polypeptide is determined. The cell, for example, can be a yeast cell or a cell of mammalian origin. Determining the ability of the test compound to bind with the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell that expresses a membrane-bound form of a polypeptide of the invention, or a biologically active portion thereof, on the cell surface with a known compound that binds to the polypeptide to form an assay mixture, contacting the assay mixture with a test compound, and determining the ability of the test compound to interact with the polypeptide, wherein determining the ability of the test compound to interact with the polypeptide includes determining the ability of the test compound to preferentially bind with the polypeptide or a biologically active portion thereof as compared to the known compound.

In another embodiment, the assay involves assessment of an activity characteristic of the polypeptide, wherein binding of the test compound to the polypeptide or a biologically active portion thereof alters (i.e., increases or decreases) the activity of the polypeptide.

Effective Dose

Toxicity and therapeutic efficacy of the molecules disclosed herein (e.g., nucleic acids, polypeptides, ribozymes, and antibodies) can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Polypeptides or other compounds that exhibit large therapeutic indices are preferred. While compounds that exhibit toxic side effects may be used, care should be taken to design a delivery system that targets such compounds to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the new methods, the therapeutically effective dose can be estimated initially from cell culture assays. A dose may be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Levels in plasma may be measured, for example, by high performance liquid chromatography.

For the compounds described herein, an effective amount, e.g. of a protein or polypeptide (i.e., an effective dosage), ranges from about 0.001 to 30 mg/kg body weight, e.g. about 0.01 to 25 mg/kg body weight, e.g. about 0.1 to 20 mg/kg body weight. The protein or polypeptide can be administered one time per week for between about 1 to 10 weeks, e.g. between 2 to 8 weeks, about 3 to 7 weeks, or for about 4, 5, or 6 weeks. Certain factors influence the dosage and timing required to effectively treat a subject, including but not limited to the type of subject to be treated, the severity of the disease or disorder, previous treatments, the general health and/or age of the subject, and other diseases present. Moreover, treatment of a subject with a therapeutically effective amount of a protein, polypeptide, antibody, or other compound can include a single treatment or, preferably, can include a series of treatments.

For antibodies, a useful dosage is 0.1 mg/kg of body weight (generally 10 mg/kg to 20 mg/kg). Generally, partially human antibodies and fully human antibodies have a longer half-life within the human body than other antibodies. Accordingly, lower dosages and less frequent administration are possible. Modifications such as lipidation can be used to stabilize antibodies and to enhance uptake and tissue penetration. A method for lipidation of antibodies is described by Cruikshank et al. ((1997) J. Acquired Immune Deficiency Syndromes and Human Retrovirology 14:193).

If the compound is a small molecule, exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Formulations and Administration

The compounds and agents, nucleic acids, polypeptides, and antibodies (all of which can be referred to herein as “active compounds”), can be incorporated into pharmaceutical compositions. Such compositions typically include the active compound and a pharmaceutically acceptable carrier or excipient. A “pharmaceutically acceptable carrier” can include solvents, dispersion media, coatings, antibacterial and antifungal agents, isotonic and absorption delaying agents, and the like, compatible with pharmaceutical administration. Supplementary active compounds can also be incorporated into the compositions.

There are a number of methods by which the new compositions for use in the new methods can be delivered to subjects, in general, and to specific cells or tissue in those subjects, in particular. In one example, plasmids encoding siRNA molecules specific to a targeted centrosomal gene can be injected into a tissue containing cells undergoing unhealthy or abnormal cell division. The plasmids would then enter cells in that tissue and express a specific siRNA, which, in turn, would silence the targeted centrosomal gene. Delivery specificity of such plasmids can be enhanced by associating them with organ- or tissue-specific affinity, so that they preferentially enter specified cell types.

Compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

The compounds may be formulated for parenteral administration by injection, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

As an another example, the compounds can be included in a topical formulation that is administered through the skin directly. Topical formulations can be applied to a region of tissue to be treated, e.g., a wound. In some cases, a topical formulation can include the use of skin permeation enhancers, such as dimethyl sulphonide (DMSO). Topical formulations can also take the form of, for example, a cream, gel, or ointment.

The compounds may also be formulated in rectal compositions such as suppositories or retention enemas, for example, containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

The therapeutic compositions of the invention can also contain a carrier or excipient, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. The nucleic acids, polypeptides, antibodies, or modulatory compounds of the invention can be administered by any standard route of administration. For example, administration can be parenteral, intravenous, subcutaneous, intramuscular, intracranial, intraorbital, opthalmic, intraventricular, intracapsular, intraspinal, intracistemal, intraperitoneal, transmucosal, or oral. The modulatory compound can be formulated in various ways, according to the corresponding route of administration. For example, liquid solutions can be made for ingestion or injection; gels or powders can be made for ingestion, inhalation, or topical application. Methods for making such formulations are well known and can be found in, for example, “Remington's Pharmaceutical Sciences”. It is expected that the preferred route of administration will be intravenous.

It is recognized that the pharmaceutical compositions and methods described herein can be used independently or in combination with one another. That is, subjects can be administered one or more of the pharmaceutical compositions, e.g., pharmaceutical compositions comprising a nucleic acid molecule or protein of the invention or a modulator thereof, subjected to one or more of the therapeutic methods described herein, or both, in temporally overlapping or non-overlapping regimens. When therapies overlap temporally, the therapies may generally occur in any order and can be simultaneous (e.g., administered simultaneously together in a composite composition or simultaneously but as separate compositions) or interspersed. By way of example, a subject afflicted with a disorder described herein can be simultaneously or sequentially administered both a cytotoxic agent which selectively kills aberrant cells and an antibody (e.g., an antibody of the invention) which can, in one embodiment, be conjugated or linked with a therapeutic agent, a cytotoxic agent, an imaging agent, or the like.

Diagnostic Methods

Compositions described herein can be used in diagnostic assays to screen for the aberrant expression of centrosomal genes. Such diagnostic assays are useful to detect (i) the presence of greater than normal centrosomal gene product in cells or (ii) the presence of less than normal centrosomal gene product in cells, e.g., in wound tissue that is not healing properly.

For example, in some methods an antibody directed to a centrosomal protein can be used to detect (i) the amount of centrosomal protein in tissue or (ii) the number of centrosomes in tissue. To detect the amount of a centrosomal protein, tissue from a subject is collected, and then protein levels in the tissue is evaluated, e.g., by Western blot, immunoprecipitation, or ELISA. These methods can be used to compare tissue from a subject with a control to determine if the subject's tissue has more or less than normal amounts of a centrosomal protein, e.g., pericentrin, δ-tubulin, ε-tubulin, γ-tubulin, GCP2, GCP3, GCP5, cNAP1, PCM1, NEK2, centrin2, CDC14A, CDC14B, gapcena, nemo, IKK1, or IKK2.

### EXAMPLES

The invention is further described in the following examples, which do not limit the scope of the invention described in the claims. The general experimental procedures are described first.

### Example 1

### siRNAs Effectively Reduce the Expression of Centrosomal Proteins and Destabilize the Centrosome

To test the ability of siRNAs to silence centrosomal gene products, cells were treated with siRNAs for 24 hours. Cells were diploid, telomerase-immortalized human retinal pigment epithelial (RPE1) cells (hTERT-RPE1s from Clontech, Palo Alto, Calif.) (see Morales et al., Nat Genet, 21:115-118, 1999). siRNAs directed to δ-tubulin (SEQ ID NO:2), γ-tubulin (SEQ ID NO:4), GCP2 (SEQ ID NO:5), GCP3(SEQ ID NO:6), cNAP1 (SEQ ID NO:8), PCM1 (SEQ ID NO:9), ninein (SEQ ID NO:21), NEK2a (SEQ ID NO:10), centrin2 (SEQ ID NO:12), CDC14A (SEQ ID NO:13), gapcena (SEQ ID NO:15), nemo (SEQ ID NO:16), pericentrin (SEQ ID NO:23), lamin A/C (CUGGACUUCCAGAAGAACA, SEQ ID NO: 24) zyxin (UGUGGCUGUCAACGAACUC, SEQ ID NO:25), and p53 (GGACAAGGGUUGGGCUGGG, SEQ ID NO:26) were made as complementary single stranded 19-mer siRNAs with 3′ dTdT overhangs (Dharmacon Inc., Lafayette, Colo.), deprotected, annealed and delivered into cells using OLIGOFECTAMINE® from Invitrogen (Carlsbad, Calif.). Nucleotides were delivered in media containing up to 100 nanomolar concentrations of siRNAs.

Commercial antibodies to the following proteins were used in this study: lamin A/C (Cell Signaling Technology, Beverly, Mass.), δ-, ε-, or γ-tubulin (Sigma-Aldrich, St. Louis, Mo.), zyxin (Santa Cruz Biotechnology, Santa Cruz, Calif.), Nek2A (Zymed, South San Francisco, Calif.), p53 (Calbiochem, San Diego, Calif.). Antibodies to pericentrin and centriolin have been described (Doxsey et al., Cell 76:639-650, 1994). Additionally antibodies to the following proteins were provided by the indicated individuals: GCP3 and GCP2 (from Tim Stearns, see Murphy et al., J. Cell Biol., 141:663-674, 1998), C-Nap1 (from Andrew Fry, see Mayor et al., J. Cell Biol., 151:837-846, 2000) Cdc14A and Cdc14B (from Peter Jackson, Nature Cell Biol., 4:318-22, 2002), ninein (from Gordon Chan, Rattner and Ou, Cell Mot. Cyto., 47:13-24 2000). Secondary antibodies were from Molecular Probes, Inc., Eugene, Oreg. To stain centrosomes, human antisera (5051) from a scleroderma subject was used. (See, e.g., Clayton et al., J. Cell Biol., 101:319-324, 1985.) This antisera recognizes multiple centrosomal proteins and is used as a general centrosomal marker.

Western blotting was performed using centrosome fractions prepared from RPE cells as described in Mitchison and Kirschner, Methods Enzymol., 134:261-268, 1986. Cells were prepared for immunofluorescence and imaged as described in Dictenberg et al., J Cell Biol., 141:163-174, 1998. Images were deconvolved using METAMORPH® software (Universal Imaging Corp., Downingtown, Pa.).

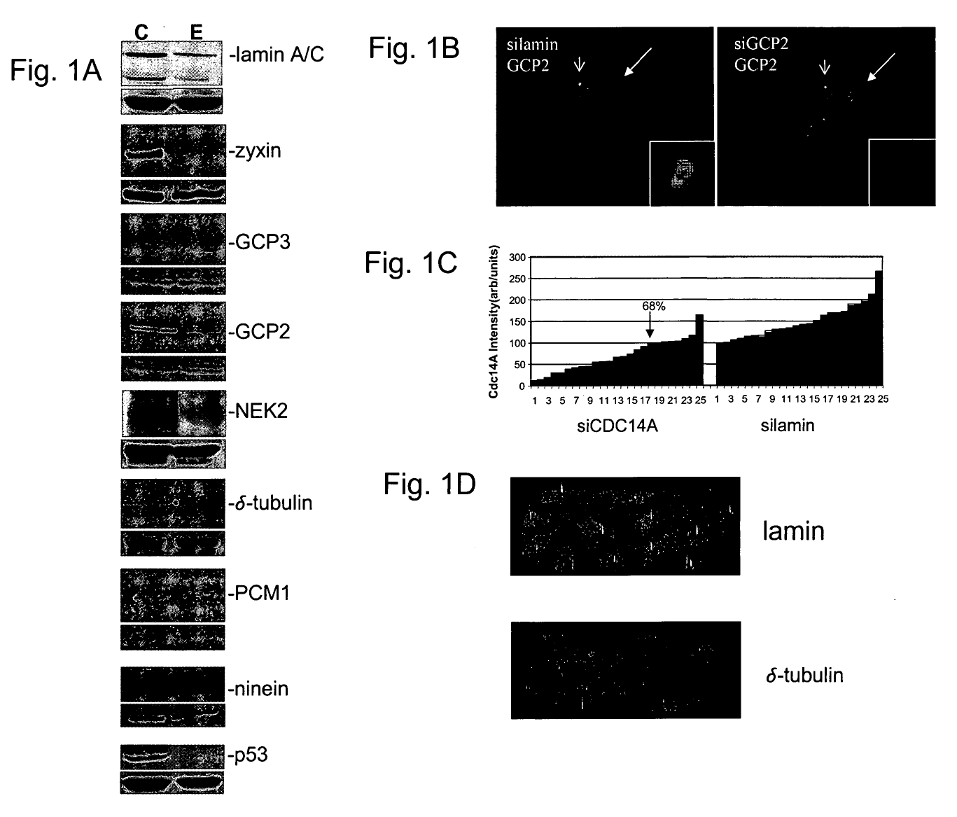

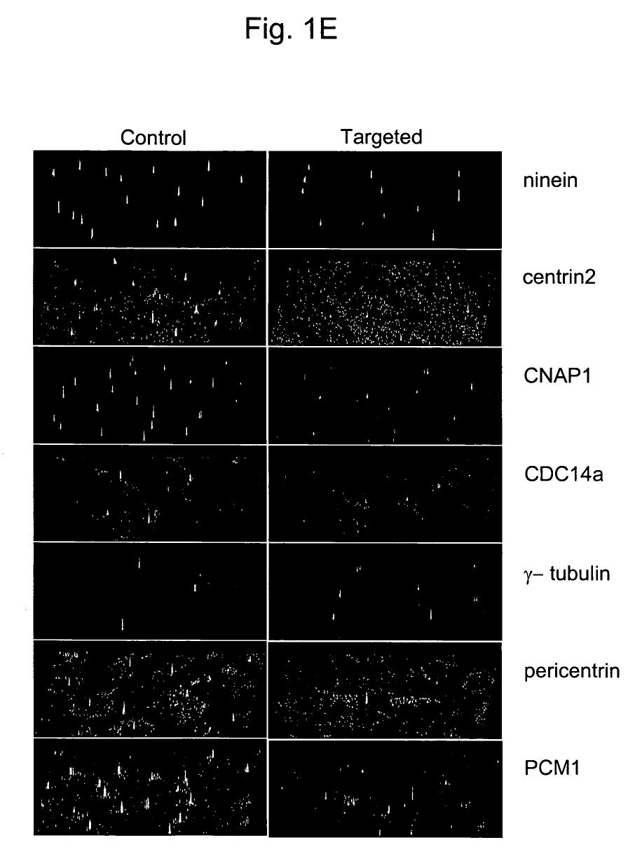

FIG. 1A depicts the specific reduction of target proteins by the indicated siRNAs. Proteins were detected by Western blot in untreated control cells (C) or siRNA-treated experimental (E) cells. For each indicated target protein, the upper panel shows an immunoblot for the target protein, and lower panel shows an immunoblot for α-tubulin to demonstrate equal loading. The blots showed that each target protein was specifically reduced.

FIG. 1B compares cells treated with siRNA to Lamin (siLamin, left panel) and GCP2 (siGCP2, right panel). Oval shaped fluorescence staining indicates nuclear DNA (large arrow). Single bright punctuate structure (small arrow) indicates the centrosome. The inset of each shows immunofluorescence staining for nuclear GCP2 protein. The two insets show that siGCP2 treatment reduced levels of GCP2 in the centrosome.

FIG. 1C is a histogram quantifying anti-cdc14A immunofluorescence observed in 25 cells treated with (i) siRNA to CDC14 (siCDC14) or (ii) control siLamin. As indicated by the graph, 68% of (i.e., 17 of 25) cells treated with siCDC14 displayed lower fluorescence intensity than the lowest control (siLamin treated) cell. These data show that siCDC14 effectively reduced expression of the CDC14 gene product.

FIG. 1D compares cells treated with siRNA to (i) δ-tubulin (siδ-tubulin) (lower panels) and (ii) lamin control (upper panel). Bright punctate staining pattern (left panel) was due to staining centrosomal δ-tubulin staining. The right hand panels show that siRNA to δ-tubulin reduced the number and/or fluorescence intensity of centrosomal staining in treated cells, relative to control cells. These data show that silencing of δ-tubulin destabilized centrosomes.

FIG. 1E extends the experiment shown in FIG. 1D to a number of other centrosomal gene products. The left hand column of panels show cells treated with control siRNA to lamin. Right hand column of panels show cells treated with siRNA directed to the indicated centrosomal gene. As in FIG. 1D, cells were stained against the siRNA targeted gene product. Control cells show endogenous levels of the protein. Staining is clearly less pronounced in right hand panels, indicating silencing of centrosomal proteins.

### Example 2

### Silencing of Centrosomal Genes Induce a G0/G1 Arrest

To further study the effects of silencing centrosomal genes, the following cell cycle experiments were performed using RPE cells. In immunofluorescence experiments, cells were delivered by OLIGOFECTAMINE in a media solution containing between 1-100 nanomolar concentration of siRNAs. After 4 hours of incubation with lipofectamine, cells were washed, and cell cycle progression was assayed by (i) treating cells with 5-Bromo-2′-deoxyuridine (BrDU from Sigma, St Louis, Mo.) for 24 hours and staining for BrDU or (ii) staining cells with antibody to Ki-67 (BD Biosciences) according to manufacturer's instructions. BrDU treatment and staining were performed as follows: after 50-70 hours incubation with siRNA, BrDU was added to cells at a final concentration of 5 μg/ml. Cells were fixed in −20° C. methanol and then treated with 2N HCL at 22° C. for 30 minutes, incubated with 1:500 mouse anti-BrdU antibody (Becton Dickinson, Palo Alto, Calif.), and labeled with 1:1,000 goat FITC-mouse antibody (Molecular Probes, Inc.). Cells were also treated with 4′,6-diamidino-2-phenylindole (DAPI) (Molecular Probes) according to the manufacturer's instructions. Cells were prepared, imaged, and the resulting images were deconvolved as described in Example 1.

Kinase assays were done on RPE cells treated with siRNAs as described above. Treated cells were lysed in 1% NP-40 lysis buffer (NP-40 buffer: 20 mM NaPO4 pH 7.4, 1% NP-40, 250 mM NaCl, 5 mM ethylenediaminetetraacetic acid (EDTA), 5 mM dithiothreitol (DTT), 1 mM phenylmethylsulfonyl fluoride, 2 μg/ml leupeptin, and 1.5 μg/ml aprotinin) supplemented with 25 mM-glycerophosphate. For measurement of CDK2-associated activity in vitro, the complexes were immunoprecipitated for 2 hours at 4° C. using anti-cylin A antibody (Santa Cruz Biotechnology). Immunocomplexes were bound to protein A-Sepharose beads for an additional hour at 4° C. and washed four times with lysis buffer followed by one wash with the kinase buffer (20 mM Tris-HCl pH 7.5, 50 mM KCl, 7.5 mM MgCl 2, 1 mM DTT, 25 mM-glycerophosphate, 1 mM phenylmethylsulfonyl fluoride, 2 μg/ml leupeptin, and 1.5 μg/ml aprotinin). Kinase reactions were performed in the presence of 2 μCi of ³²P-ATP for 15 min at 30° C. using 4 μg of histone H1 as substrates in kinase buffer. Phosphorylated proteins were analyzed on 10% SDS-polyacrylamide gels followed by autoradiography and quantified by phosphorimaging (Molecular Imager GS-250; Bio-Rad, Hercules, Calif.).

For flow cytometry (i.e., fluorescence-activated cell sorter or FACS) experiments, siRNAs were delivered to cells as described above, and were incubated for 50-70 hours. To synchronize cells, nocodazole was added to the cells to a concentration of 5 μg/mL for 12 hours. Cells were stained with propidium iodide and analyzed by flow cytometry (FACSCAN; Becton Dickinson) using FLOJO® software (Tree Star, Inc., Ashland, Oreg.).

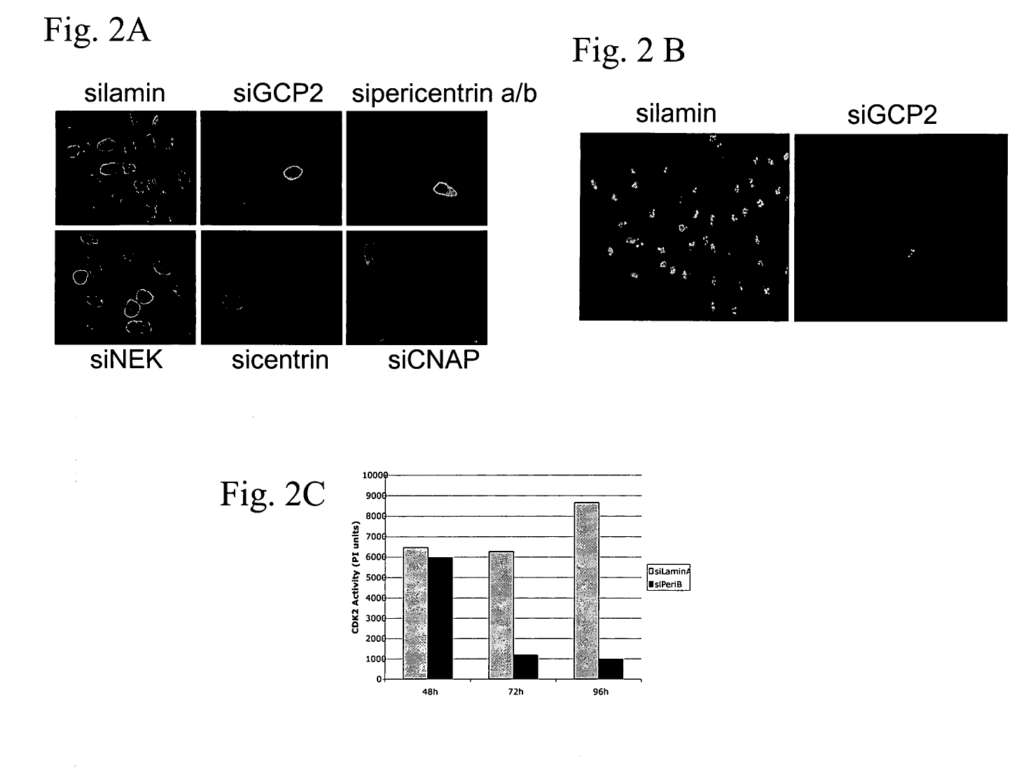

Each panel of FIG. 2A shows cells treated with the indicated siRNA and synchronized using nocodazole. Upon release from nocodazole treatment, proliferating cells typically resume cell cycle progression. Chromosomal DNA in the cells was imaged using DAPI (faint ghost-like staining) and/or BrDU (brighter staining). Although DAPI can be used to visualize DNA at any stage of the cell cycle, BrDU is only incorporated into newly synthesized DNA only during S-phase. Thus, BrDU incorporation can be used as a marker of cells that progress through at least one S-phase of the cell cycle. Control cells in this experiment were treated with siRNA to lamin. As the upper left hand panel in FIG. 2A shows, nearly every control cell resumed cell cycle progression and incorporated BrDU. FIG. 2A also shows that siRNA-mediated silencing of GCP2, pericentrin, centrin, and CNAP clearly inhibited incorporation of BrDU into chromosomal material, relative to control cells. The effect of siRNA directed to NEK appears to be less pronounced (perhaps due to inefficient silencing of the gene). These data indicate that silencing of at least some centrosomal gene products causes cell cycle arrest that can prevent even one progression through the cell cycle.

FIG. 2B confirms the results in FIG. 2A using a different immunofluorescence marker for cell cycle progression. Ki67 is a nuclear protein marker of proliferating cells that is largely absent from non-proliferating cells resting in G0/G1 phase of the cell cycle. DAPI nuclear staining can be seen as faint ghost-like nuclear staining in both panels of FIG. 2B. Cells treated with silamin (left panel) displayed a large number of bright punctate staining with antibodies to Ki67. Very few cells treated with siGCP2 (right panel), on the other hand, express Ki67. These results indicate that depletion of at least some centrosomal proteins can cause cells to stop proliferating, by arresting them in the G0/G1 phase of the cell cycle.

FIG. 2C is a graph quantifying the results of CDK2 kinase assays from cells treated with siRNA directed to laminin or pericentrin B. CDK2 activity is required for proliferating cells through to progress through the G1 stage of the cell cycle. The data shown in FIG. 2C indicates that CDK2 expression and/or activity was greatly diminished after sustained treatment with siRNA to pericentrin B. These data, consistent with other experiments described herein, show that depletion of a centrosomal protein induces a cell cycle arrest at a stage that precedes exit from the G1 phase.

The absence of BrDU incorporation into cells depleted for a centrosomal gene product (described in a number of Examples) is significant for indicating that these cells do not arrest after duplicating chromosomal material. This inability to duplicate chromosomal material means that depletion of centrosomal proteins does not result in polyploidy or multinucleated cells, which are associated with multiple cellular disorders.

### Example 3

### Depletion of Centrosomal Gene Products Inhibits Cell Proliferation

In another approach to evaluating the effect requirement for centrosomal gene products, fluorescence assisted cell sorting (flow cytometry or FACS) analysis was performed as described in Gromley et al., J. Cell Biol., 161:535-545 (2003). Briefly, cells were treated with indicated siRNA for at least 50-70 hours, unless otherwise indicated. Cells were synchronized by adding nocodazole to 5 μg/mL for 12 hours prior to cell collection. Cells were collected, fixed in methanol, stained with propidium iodide, and analyzed by flow cytometry (FACSCAN®;Becton Dickinson) using FLOJO® software (Tree Star, Inc.).

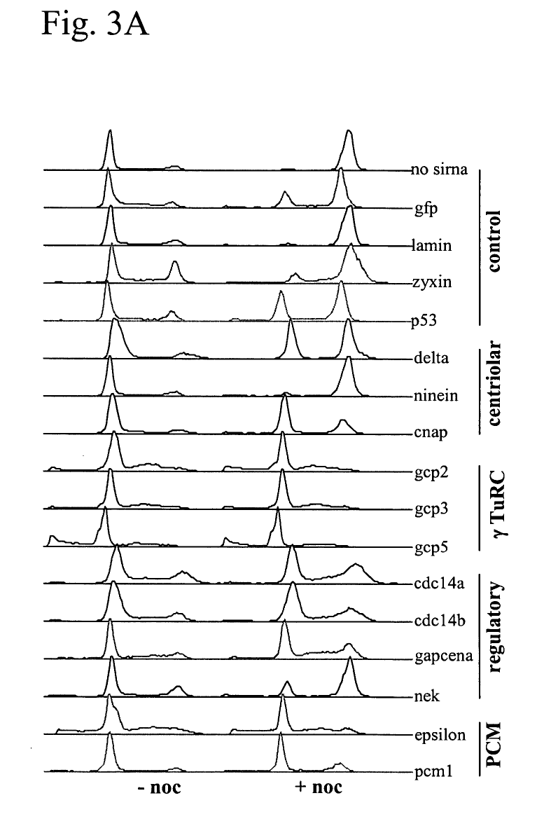

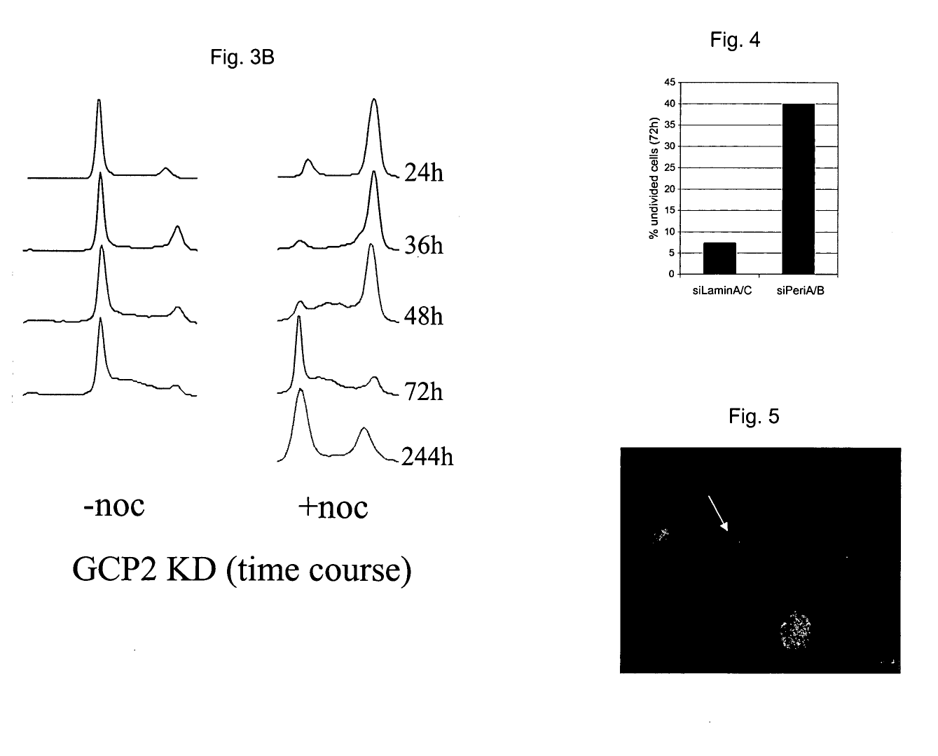

FIG. 3A is a graph showing the effects of depleting the indicated centrosomal gene products by siRNA treatment. The left side of the graph depicts the FACS results for treated cells before the addition of nocodazole (-noc). The major fluorescence peak in (-noc) cells is the G1 peak, (i.e., lower DNA fluorescence). The right side of the graph depicts the results for cells after nocodazole treatment (+noc). In control cells, synchronization with nocodazole causes a pronounced shift in the fluorescence peak to a G2/M peak (i.e., DNA fluorescence roughly doubles relative to the G1 peak). siRNas directed to ninein and NEK did not cause a pronounced inhibition of the shift from G1 to G2/M peak. This may be due to inefficient silencing caused by the siRNAs used. Nonetheless, the data also show that depletion of the other centrosomal gene products tested effectively inhibited the nocodazole-induced shift seen in the majority of control cells. These results indicate that centrosome proteins are required for cell cycle progression, and that depletion of at least some centrosomal proteins causes cell cycle arrest at G0/G1 phase.

FIG. 3B is a graph showing the time course of siRNA-induced cell cycle arrest. All cells were treated with siRNA to GCP2 for the indicated number of hours. The left side of the graph depicts FACS results for cells before the addition of nocodazole (−noc), and right hand depicts FACS results for cells after addition of nocodazole (+noc). The data indicate that prevention of the nocodazole shift does not occur until some time after 48 hours of siRNA treatment. This may be due to an absence of significant turnover of GCP2 protein in the centrosome during the initial 48 hours of siRNA treatment, such that only after 48 hours does depleting nascent GCP protein cause a centrosomal defect. On the other hand, by 72 hours, siRNA treatment effectively blocked the nocodazole-induced shift to G2/M peak. The data further show that this block lasts for at least 244 hours. Thus, these results indicate that inhibition of a centrosomal protein brings about a lasting block of cell proliferation. The results are also consistent with the notion that depletion of centrosomal proteins can induce a differentiated, non-proliferative state in previously proliferative cells.

### Example 4

### Depletion of Pericentrin Inhibits Cell Division

RPE cells were transfected with siRNA directed to pericentrin A/B or lamin (control) as described in Example 2. Immediately prior to siRNA transfection cells were labeled with a succinimidyl ester of carboxyfluorescein diacetate (CFDA-SE from Molecular Probes, Eugene, Oreg.) according to manufacturers instructions. CFDA-SE is a fluorescent marker that spontaneously and irreversibly couples to free amines on intracellular and cell-surface proteins. When a cells divides, CFDA-SE labeling is inherited equally by daughter cells, which are therefore half as fluorescent as the parent cell. Cells were prepared for and analyzed by fluorescence microscopy as described in Example 1.

FIG. 4 is a histogram showing that, during the 72 hours immediately following treatment with siRNA to pericentrin, 40% never divided. When compared to control cells, in which only ˜7% of cells did not divide, these data confirm that inhibition of a centriolar gene product imposes cell cycle arrest, which severely inhibits the cell division of previously proliferative of cells.

### Example 5

### Transfection of a Pericentrin Polypeptide Fragment Induces Cell Cycle Arrest

As an alternative way of inhibiting centriolar gene product function, a PACT domain containing pericentrin fragment was overexpressed in RPE cells. The PACT domain is responsible for targeting pericentrin to the centrosome. The pericentrin fragment included the carboxyl (c)-terminal 241 amino acids of pericentrin, which were fused to the c-terminus of GFP. This GFP chimera was expressed from a plasmid vector described in Gillingham et al., EMBO Rep., 1:524-9 (2000). RPE cells were synchronized with 5 ug/mL nocodazol and microinjected upon release. Microinjection was done with an Eppendorf transjector 5246 and Micromanipulator (Brinkmann, Westbury, N.Y.). Plasmids were introduced via intranulclear injection at concentrations of 200 ng GFP-PERICT1. Following injection cells incubated with 5 ug/mL BrDU for 24 hours and processed for immunofluorescence.

FIG. 5 shows that at least some BrDU incorporated into the chromosomes of most cells in the image except for the cell (arrow) expressing the transgene. The image also shows that the PACT domain was targeted to the centrosome (pair of perinuclear red dots in cell indicated by the arrow. Since BrDU was not effectively taken up in transfected cells, the results indicate that the targeting domain of a centrosomal protein can be used to arrest cell cycle of a previously proliferative cell.

### Example 6

### Cell Cycle Arrest Phenotype is p53 Dependent

A number of experiments were conducted to determine if p53 is required for the cell cycle arrest phenotype described herein. FACS analysis of tandem siRNA-treated cells was performed as described in Example 2. BrDU and DAPI imaging was performed as described in Example 2. Intracellular p53 was visualized using anti-p53 antibody that is specific for activated p53 (i.e., p53 phosphorylated at Serine 33) (“α-Pser33-p53” provided by Yoichi Taya and described in Kishis et al., J. Biol. Chem., 276:39115-3922, 2001)

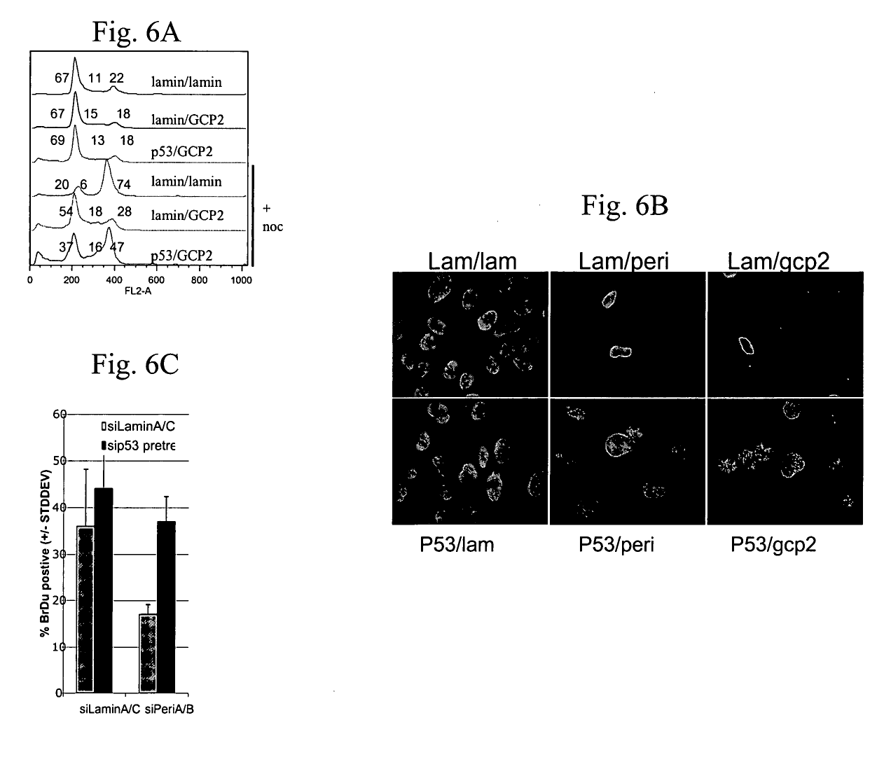

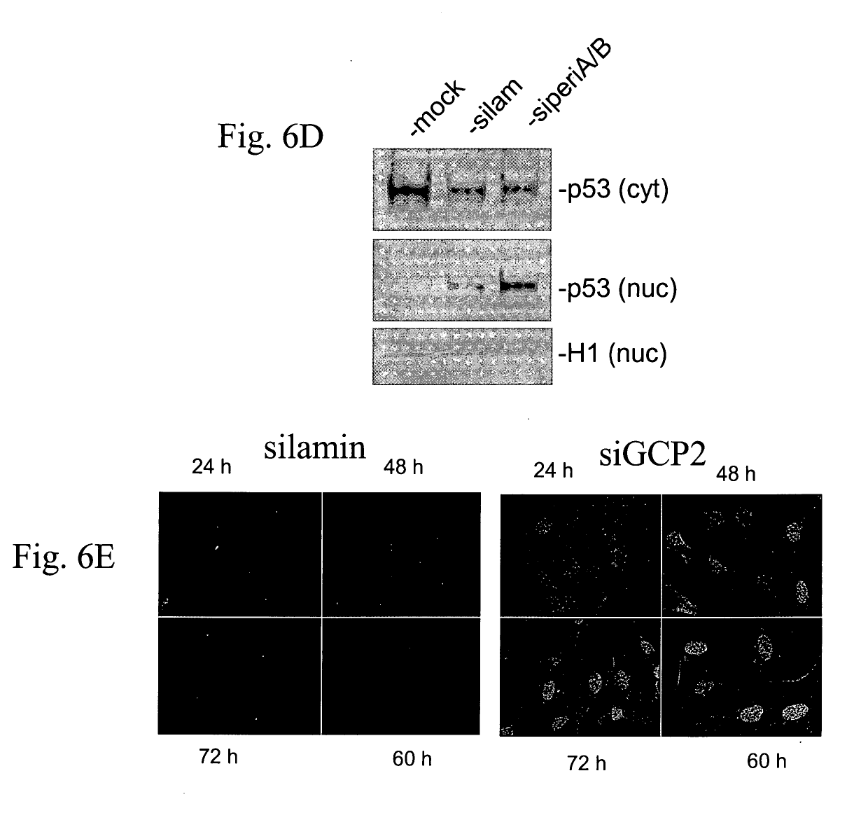

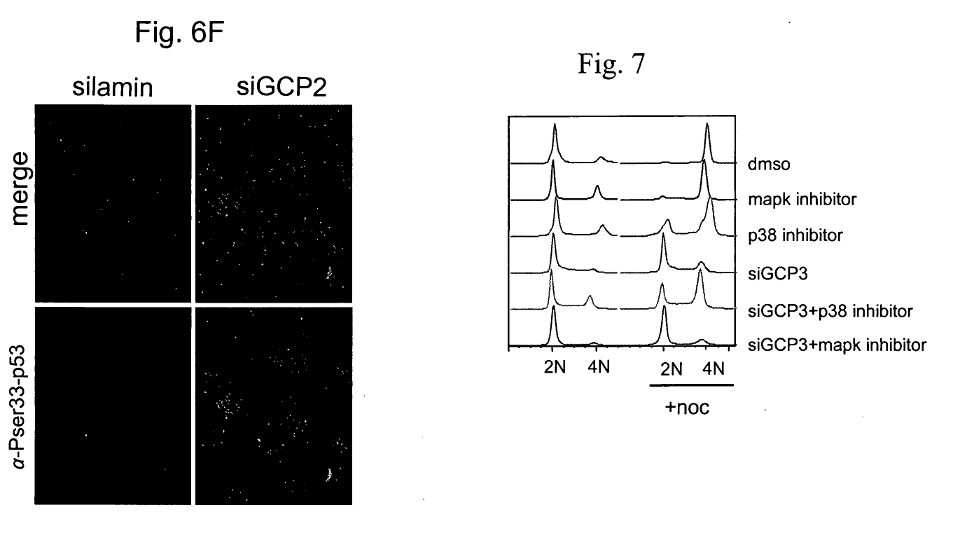

In FIG. 6A each line tracing represents FACS data from cells treated in tandem with the siRNAs directed to the gene products indicated above the line. Cells were first treated with siRNA directed to the gene product to the left of the slash. Up to 24 hours later, siRNA directed to the gene product to the right of the slash was added. Cells were treated with siRNA for up to 72 hours. 12 hours before collecting the cells, nocodazole was added to the cells analyzed in the bottom three line tracings. Control (silamin/silamin treated) cells, displayed the expected nocodazole shift from G1 peak to G2/M peak. This shift was blocked in cells treated in tandem with silamin and siGCP2. However, pre-treatment with siRNA to p53 inhibited the siGCP-mediated block of the nocodazole shift. In other words, depletion of p53, at least partially, suppressed the cell cycle arrest phenotype associated with depletion of GCP2 gene product. These results suggest that GCP2 mediated cell cycle arrest is dependent on p53.

FIG. 6B shows BrDU staining of cells treated in tandem with the indicated siRNAs. Upper left hand corner shows that a majority of control (silamin/silamin treated) cells incorporated BrDU into their chromosomal, indicating that the cells divided at least once. As expected, substantially fewer silaminin/sipericentrin or silaminin/siGCP2 treated cells incorporated BrDU, indicating that these cells did not divide. Pretreatment of cells with sip53, however, increased the number of cells that divided even after being treated with sipericentrin or siGCP2. Some of these data are quantified by the graph depicted in FIG. 6C. These results confirm that p53 is required for depletion of centrosomal gene product to cause cell cycle arrest in previously proliferative cells.

FIG. 6D is a Western blot showing that nuclear p53 is activated in cells treated with sip53, relative to mock transfected cells or silamin transfected cells. The Western blot shown cytoplasmic (cyt), and nuclear (nuc) cell fractions blotted with an antibody to p53 (from Callbiochem, San Diego, Calif.). Nuclear fractions were blotted with an antibody for H1 as a control for loading. These results suggest that silencing of centrosomal gene product causes translocation of p53 from the cytoplasm into the nucleus.

Translocation of p53 into the nucleus caused by depletion of centrosomal proteins was also observed by immunofluorescence. FIG. 6E shows p53 expression in cells treated with silamin (left-hand panels) or siGCP2 (right hand panels) for the indicated amounts of time. Relatively little p53 translocated from the cytoplasm to the nucleus of silamin. In siGCP2-treated cells, on the other hand, substantially more p53 accumulated in nucleus. These results also indicate that depletion of a centrosomal gene product causes translocation of p53 from the cytoplasm into the nucleus.

An antibody specifically reactive for phosphorylated p53 (α-Pser33-p53) was used to determine the phosphorylated state of p53 that accumulates in the nucleus as a result of GCP2 gene product depletion. In FIG. 6F, the left panels depict cells treated with silamin; the right panels depict cells treated with siGCP2. The two bottom panels shows cell staining with α-Pser33-p53 antibody. The two upper panels show a merged view of (i) the bottom panels and (ii) the same cells stained with DAPI. As can be seen in the bottom panels, activated p53 was detected only in the nucleus of cells treated with GCP2. This nuclear localization is confirmed by the overlapping DAPI staining.

### Example 7

### Cell Cycle Arrest is p38-Dependent

The following experiment was performed to test if p38, an upstream component of p53 signaling, is required for the centrosomal cell cycle arrest phenotype described herein. The following inhibitors were added to RPE1 cells 1 hour prior to siRNA treatment at the indicated concentrations: SB203580 (p38 inhibitor) 40 μM, SB202190 (p38 inhibitor) 10 μM, and PD98059 (MAPK inhibitor) 50 μM. All inhibitors were obtained from Calbiochem and dissolved in DMSO. siRNA treatment and FACS analysis was performed as described in Example 2.

The three upper line tracings in FIG. 7 show that treatment with a map kinase inhibitor and p38 inhibitors did not inhibit the nocodazole-induced shift in cells from G1 to G2/M peak that is typically seen in proliferative cells. Treatment with siGCP3 inhibited this shift, demonstrating that depletion of GCP3 causes cell cycle arrest. Combined treatment with map kinase inhibitor and siGCP3 did not affect siGCP3's ability to cause cell cycle arrest. In experiments that are not shown, it was also determined that inhibitors of JNK kinase did not affect siGCP3's ability to inhibit cell cycle progression. However, pre-treatment with p38 inhibitors dramatically reduced siGCP3's inhibitory potency, as can be seen by the noticeable shift in cells from the G1 peak to G2/M peak.

These results are consistent with a model of cell cycle arrest that includes the following metabolic steps: (i) depletion of a centrosomal protein (ii) activation of p38, and (iii) activation of p53.

### Example 8

### Depletion of Multiple Centrosomal Gene Products Cause Cell Cycle Arrest

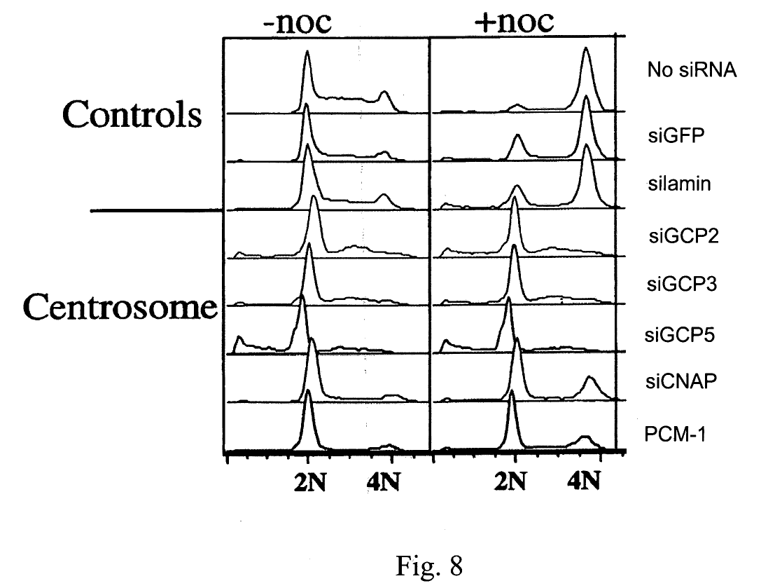

The ability of a number of centrosomal genes to inhibit cell cycling of RPE1 cells was tested by treating cells with siRNAs and performing FACS analysis as described in Example 2. FIG. 8 shows that siRNA mediated depletion of GCP-2, GCP-3, GCP-5, CNAP, and PCM-1 prevented nocodazole-induced shift from the G1 peak to G2/M peak. These results show that inhibition of each one of these proteins can be used to impose a cell cycle arrest on previously proliferative cells.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of reducing cell division in a subject, the method comprising administering to the subject a composition in an amount sufficient to decrease cell division in the subject, wherein the composition inhibits function or expression of a centrosomal gene product selected from the group consisting of δ-tubulin, ε tubulin, γ-tubulin, GCP2, GCP3, GCP5, cNAP1, PCM1, centrin2, CDC14A, CDC14B, gapcena, and nemo.

2. The method of claim 1, wherein the composition is an siRNA.

3. The method of claim 1, wherein the composition comprises a sequence selected from the group consisting of SEQ ID NOs:2-22 and 24-26.

4. The method of claim 1, wherein the composition is an antibody.

5. The method of claim 1, wherein the composition is an antibody that binds to a protein selected from the group consisting of δ-tubulin, ε-tubulin, γ-tubulin, GCP2, GCP3, GCP5, cNAP1, PCM1, centrin2, CDC14A, CDC14B, gapcena, and nemo.

6. The method of claim 1, wherein the subject is suffering from a carcinoma, tumor, psoriasis, leukemia, Hodgkin's disease, lymphoma, myelofibrosis, or polycythemia vera

7. A method of identifying an anti-proliferative compound, the method comprising:
selecting a test compound that inhibits activity or localization of a centrosomal gene product, wherein the gene product is selected from the group consisting of δ-tubulin, ε-tubulin, γ-tubulin, GCP2, GCP3, GCP5, cNAP1, PCM1, centrin2, CDC14A, CDC14B
contacting the compound to a cell under conditions which induce the cell to proliferate when the test compound is absent; and
determining if the test compound induces cell cycle arrest, wherein a test compound that induces cell cycle arrest is an anti-proliferative compound.

8. The method of claim 7, wherein the method further includes:
screening a test compound to determine if it binds to a centrosomal gene or a polypeptide encoded by a centrosomal gene, wherein the gene encodes a gene product selected from the group consisting of δ-tubulin, ε-tubulin, γ-tubulin, GCP2, GCP3, GCP5, cNAP1, PCM1, centrin2, CDC14A, CDC14B, gapcena, and nemo; and
selecting the test compound as a compound that inhibits activity or localization of a centrosomal gene product, if the test compound binds to the gene or the polypeptide.

9. The method of claim 7, wherein the test compound is an siRNA.

10. The method of claim 7, wherein the test compound is an antisense molecule.

11. The method of claim 7, wherein the test compound is an antibody

12. A composition that inhibits function or expression of a centrosomal gene product, wherein the gene product is selected from the group consisting of δ-tubulin, ε tubulin, γ-tubulin, GCP2, GCP3, GCP5, cNAP1, PCM1, centrin2, CDC14A, CDC14B, gapcena, and nemo.

13. The composition of claim 12, wherein the composition is an siRNA.

14. The composition of claim 12, wherein the composition is an antisense RNA

15. The composition of claim 12, wherein the composition is a polypeptide fragment of the centrosomal gene product.

16. The composition of claim 15, wherein the fragment comprises a centrosomal targeting domain of the centrosomal gene product.

17. A method of reducing cell division in a subject, the method comprising administering to the subject the composition of claim 16.

18. A pharmaceutical composition comprising a centrosomal targeting domain of pericentrin.

19. A method of reducing cell division in a subject, the method comprising administering to the subject the composition of claim 18.

20. An isolated nucleic acid molecule comprising a nucleotide sequence selected from the group consisting of SEQ ID NOs:2-22 and 24-26.


SEQUENCE LISTING (26 sequences)

SEQ ID NO: 1 - RNA, 19 nt, Artificial Sequence
  Synthetically generated siRNA
gcagcugagcugaaggaga

SEQ ID NO: 2 - RNA, 19 nt, Artificial Sequence
  Synthetically generated siRNA
gguucuggaaacaacuggg

SEQ ID NO: 3 - RNA, 19 nt, Artificial Sequence
  Synthetically generated siRNA
agucggcagagcacuguga

SEQ ID NO: 4 - RNA, 19 nt, Artificial Sequence
  Synthetically generated siRNA
ugaccgcaaggacgucuuu

SEQ ID NO: 5 - RNA, 19 nt, Artificial Sequence
  Synthetically generated siRNA
ucucguacuccagaagacu

SEQ ID NO: 6 - RNA, 19 nt, Artificial Sequence
  Synthetically generated siRNA
aagagaagcagaugcugca

SEQ ID NO: 7 - RNA, 19 nt, Artificial Sequence
  Synthetically generated siRNA
acuucgccugguccaacuu

SEQ ID NO: 8 - RNA, 19 nt, Artificial Sequence
  Synthetically generated siRNA
ucuauccgaaagcccaguc

SEQ ID NO: 9 - RNA, 19 nt, Artificial Sequence
  Synthetically generated siRNA
gucccccaaacagagaaac

SEQ ID NO: 10 - RNA, 19 nt, Artificial Sequence
  Synthetically generated siRNA
ucagcuucgugauucucag

SEQ ID NO: 11 - RNA, 19 nt, Artificial Sequence
  Synthetically generated siRNA
agggaaccaaggaaaggca

SEQ ID NO: 12 - RNA, 19 nt, Artificial Sequence
  Synthetically generated siRNA
gagcaaaagcaggagaucc

SEQ ID NO: 13 - RNA, 19 nt, Artificial Sequence
  Synthetically generated siRNA
gcacaguaaauacccacua

SEQ ID NO: 14 - RNA, 19 nt, Artificial Sequence
  Synthetically generated siRNA
gcaaaugcugccuuccuug

SEQ ID NO: 15 - RNA, 19 nt, Artificial Sequence
  Synthetically generated siRNA
ccagagaugagccuaccag

SEQ ID NO: 16 - RNA, 19 nt, Artificial Sequence
  Synthetically generated siRNA
gagaugccagcagcagaug

SEQ ID NO: 17 - RNA, 19 nt, Artificial Sequence
  Synthetically generated siRNA
acagagaacgauggugcca

SEQ ID NO: 18 - RNA, 19 nt, Artificial Sequence
  Synthetically generated siRNA
ucaggaaacaggugagcag

SEQ ID NO: 19 - RNA, 19 nt, Artificial Sequence
  Synthetically generated siRNA
ggaucagagacucuaccuu

SEQ ID NO: 20 - RNA, 19 nt, Artificial Sequence
  Synthetically generated siRNA
gugcugcagcagacauuac

SEQ ID NO: 21 - RNA, 19 nt, Artificial Sequence
  Synthetically generated siRNA
uaugagcauugaggcagag

SEQ ID NO: 22 - RNA, 19 nt, Artificial Sequence
  Synthetically generated siRNA
cugucacucaagccaagga

SEQ ID NO: 23 - RNA, 19 nt, Artificial Sequence
  Synthetically generated siRNA
uuggaacagcugcagcaga

SEQ ID NO: 24 - RNA, 19 nt, Artificial Sequence
  Synthetically generated siRNA
cuggacuuccagaagaaca

SEQ ID NO: 25 - RNA, 19 nt, Artificial Sequence
  Synthetically generated siRNA
uguggcugucaacgaacuc

SEQ ID NO: 26 - RNA, 19 nt, Artificial Sequence
  Synthetically generated siRNA
ggacaaggguugggcuggg